(19) United States
(12) Patent Application Publication
(10) Pub. No.: US 2026/0078170 A1
(43) Pub. Date: Mar. 19, 2026

(54) Pan-Neutralizing SARS-CoV-2 mAb Composition and Methods of Treatment Thereof

(71) Applicant: Washington University, St. Louis, MO (US)

(72) Inventor: Ali Ellebedy, St. Louis, MO (US)

(73) Assignee: Washington University, St. Louis, MO (US)

(21) Appl. No.: 19/078,046

(22) Filed: Mar. 12, 2025

Related U.S. Application Data
(60) Provisional application No. 63/564,476, filed on Mar. 12, 2024.

(51) Int. Cl. C07K 16/10; A61K 39/00; A61K 39/215; A61P 35/00

(52) U.S. Cl. CPC C07K 16/104; A61K 39/215; A61P 35/00; A61K 2039/505; A61K 2039/53; A61K 2039/545; C07K 2317/565; C07K 2317/76

(57) ABSTRACT

Among the various aspects of the present disclosure is the provision of compositions and methods of use of mAb that prevent, inhibit, or reduce the transmissivity of SARS-CoV-2 infections.

### CROSS-REFERENCE TO RELATED APPLICATIONS

This application claims the benefit of priority to U.S. Provisional Application Ser. No. 63/564,476 filed on Mar. 12, 2024, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention (file name “020863-US—NP_sequence listing” created on 6 Mar. 2025; 19,161 bytes). The subject matter of the Sequence Listing is incorporated herein by reference in its entirety.

### FIELD OF THE INVENTION

The present disclosure generally relates to mAbs, SARS-CoV-2 vaccines comprising the mAbs, and methods of preventing SARS-CoV-2 infections.

### BACKGROUND OF THE INVENTION

Viral infections are responsible for hundreds of thousands of deaths each year. However, treatment options are limited for many viruses. Additionally, carriers of a virus may be asymptomatic, leading to high transmission rates from infected but asymptomatic individuals.

Severe acute respiratory syndrome coronavirus 2 (SARS-CoV-2) is the etiologic agent of the Coronavirus Disease 2019 (COVID-19) syndrome, which can rapidly progress to pneumonia, respiratory failure, and systemic inflammatory disease. SARS-CoV-2 is a positive-sense single-stranded RNA virus that was first isolated in late 2019 from patients with severe respiratory illness in Wuhan, China. As a beta-coronavirus, SARS-CoV-2 is related to two other highly pathogenic respiratory viruses, SARS-COV and Middle East respiratory syndrome coronavirus (MERS-COV). SARS-CoV-2 infection results in a clinical syndrome that can progress to respiratory failure and also present with cardiac pathology, gastrointestinal disease, coagulopathy, and hyperinflammatory syndrome. The elderly, immunocompromised, and those with co-morbidities (e.g., obesity, diabetes, and hypertension) are at greatest risk of death from COVID-19. More than 152 million infections and 3.2 million deaths have been recorded worldwide since the start of the pandemic.

Although various vaccines have substantially reduced the overall transmission and severity of symptoms associated with COVID-19, SARS-CoV-2 continues to evolve escape variants that are less susceptible to existing vaccine compositions, necessitating the continual development of updated vaccine compositions capable of targeting the escape variants as they arise,

A need exists for the development of a vaccine targeting a highly conserved region of the SARS-CoV-2 virus that is less prone to mutating into an escape variant.

### SUMMARY OF THE INVENTION

Briefly, therefore, the present disclosure is directed to vaccine compositions to prevent or reduce the transmissivity and symptoms of an infection by the SARS-CoV-2 virus.

In one aspect, a composition to treat a SARS-CoV-2 infection that includes an mAb-52 monoclonal antibody is disclosed. In some aspects, the composition further comprises a SARS-CoV-2 mRNA vaccine and a pharmaceutically acceptable carrier. In some aspects, the SARS-CoV-2 mRNA vaccine is mRNA-1273.213, mRNA-1273.214, mRNA-1273.529, BNT162b2, and any combination thereof. In some aspects, the mAb-52 monoclonal antibody comprises a heavy chain, IGHV3-66, with the sequence
MTWVRQAPGKGLEWVSLIFAGGSTFYADSVKGRFTVSRDNSKNTLYLQMSSLK PEDTAVYFCARDLREMGGLDFWGQGALVTVSS (SEQ_ID_NO: 3) and variants thereof and a light chain with the sequence
MTQSPSSVSASIGDTVTITCRASQGIPSWVAWYQQKPGKAPKLLIYGASNLQRG VPSRFSGSGSGTDFTLTISSLOPEDLAVYYCHQSDSLPGIFGGGTKVEIK (SEQ_ID_NO: 4) and variants thereof. In some aspects, the mAb-52 monoclonal antibody targets a receptor binding domain (RBD) of the SARS-CoV-2 spike protein and competes with ACE2. In some aspects, the mAb-52 targets a class I/II RBD epitope of the SARS-CoV-2 spike protein. In some aspects, the mAb-52 binds the RBD of the SARS-CoV-2 spike protein with interactions comprising polar and hydrophobic interactions of CDRH1, CDRH2, CDRH3, CDRL1, and CDRL3. In some aspects, the mAb-52 neutralizes a SARS-CoV-2 strain comprising the variants WA1, BA.1, XBB.1.5, EG.5.1, BA.2.86, HV.1, JN.1, and KP.2.

In another aspect, a method of treating a SARS-CoV-2 infection is disclosed that includes administering a therapeutically effective amount of a composition comprising an mAb-52 monoclonal antibody. In some aspects, the composition further comprises a SARS-CoV-2 mRNA vaccine and a pharmaceutically acceptable carrier thereof. In some aspects, the SARS-CoV-2 mRNA vaccine is mRNA-1273.213, mRNA-1273.214, mRNA-1273.529, BNT162b2, and any combination thereof. In some aspects, the mAb-52 comprises a heavy chain, IGHV3-66, with the sequence
MTWVRQAPGKGLEWVSLIFAGGSTFYADSVKGRFTVSRDNSKNTLYLQMSSLK PEDTAVYFCARDLREMGGLDFWGQGALVTVSS (SEQ_ID_NO: 3) and variants thereof, and a light chain with the sequence
MTQSPSSVSASIGDTVTITCRASQGIPSWVAWYQQKPGKAPKLLIYGASNLQRG VPSRFSGSGSGTDFTLTISSLOPEDLAVYYCHQSDSLPGIFGGGTKVEIK (SEQ_ID_NO: 4) and variants thereof. In some aspects, the mAb-52 monoclonal antibody is administered to the subject at a dose ranging from 5 mg/kg to 15 mg/kg. In some aspects, the mAb-52 monoclonal antibody is administered at a dose of 10 mg/kg. In some aspects, the mAb-52 monoclonal antibody targets a receptor binding domain (RBD) of the SARS-CoV-2 spike protein and competes with ACE2. In some aspects, the mAb-52 monoclonal antibody targets a class I/II RBD epitope of the SARS-CoV-2 spike protein. In some aspects, the mAb-52 monoclonal antibody neutralizes a SARS-CoV-2 strain comprising the variants WA1, BA.1, XBB.1.5, EG.5.1, BA.2.86, HV.1, JN.1, and KP.2.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic of an experimental timeline. Nine participants previously immunized with three doses of ancestral WA1/2020 (WA1) vaccine were enrolled and given a fourth WA1 booster dose. All participants' blood was collected at baseline, week 1, 4, 8 and 17 following boosting. Fine needle aspirate of draining axillary lymph nodes (LN FNA) were collected from all participants at week 8 post-boosting.

FIG. 1B is a graph of the frequency of bivalent vaccine WA1 and BA.1 S+ antibody responses probed by ELISpot at week 1 corresponding to peak plasmablast (PB) response time-point.

FIG. 1C is a set of graphs of plasma anti-S IgG titers measured at week 0 and week 4 against WA1 (left) and BA.1 (right) Spike protein (n=9). Results are from technical duplicates of one experiment. P values were determined by two-tailed Wilcoxon matched-pairs signed rank test.

FIG. 1D is a set of flow cytometry plots (top) and a corresponding graph (bottom) of S-binding germinal center B cells (BCL6+CD38intlgDloCD19+CD3−) of fine needle aspirates from draining axillary lymph nodes at week 8 post boosting. Horizontal line on the bottom graph indicates the median.

FIG. 1E is a set of flow cytometry plots (top) and a corresponding graph (bottom) of S-binding germinal center B cells (CD20loCD38+IgDloCD19+CD3−) of fine needle aspirates from draining axillary lymph nodes at week 8 post boosting. Horizontal line on the bottom graph indicates the median.

FIG. 2A is a set of uniform manifold approximation and projection (UMAP) plots representing B cell transcriptional clusters from single cell RNA sequencing (scRNA-seq) of lymph node FNAs (left) with spike-specific clones overlaid (right). Each dot represents a cell, colored by phenotype as defined by transcriptomic profile (left) and S-specificity (right). Total number of cells (left) and number of S+ cells (right) are on the top left corner. GC, GC B cell; LNPC, Lymph node plasma cell; MBC, Memory B cell.

FIG. 2B is a pie chart of the antigen binding landscape of GC B cell and LNPC mAbs (n=598) derived from distinct B cell clonal lineages from lymph node FNA at week 8 of five participants.

FIG. 2C is a pie chart of the binding of mAbs from S-specific GC B cells and LNPCs at day 57 post boosting to constituent domains of WA1/2020 S protein measured by ELISA.

FIG. 2D is a graph WA1/2020 RBD-binding mAbs binding variants of concern RBD. The number of mAbs binding RBD of WA1/2020; WA1/2020 and BA.1; and WA1/2020, BA.1, and XBB.1.5 are depicted.

FIG. 2E is a plot of the neutralizing activity of mAbs cross reacting with WA1/2020, BA.1, and XBB.1.5 as determined with VSV-S (WA1/2020 D614G) chimeric virus assays. Each symbol represents one mAb. Percentages indicate the proportion of mAbs above 90% infection reduction threshold.

FIG. 2F is a plot of the neutralizing activity of mAbs in infection against WA1/2020 D614G, BA.1, XBB.1.5, EG.5.1, BA.2.86, HV.1, JN.1 authentic viruses. Each symbol represents one mAb. Authentic virus neutralization IC50 quantitated in ng/ml and mAbs are considered neutralizing given IC50<1000 ng/ml. mAb-52 potently neutralized WA1 D614G, BA.1, XBB.1.5, EG.5.1, BA.2.86, HV.1, JN.1 viral variants. Results are from technical duplicates of one experiment.

FIG. 2G is a plot of the neutralizing activity of mAbs in infection against KP.2 authentic virus. Each symbol represents one mAb. Authentic virus neutralization IC50 quantitated in ng/ml and mAbs are considered neutralizing given IC50<1000 ng/ml. mAb-52 potently neutralized KP.2 viral variants. Results are from technical duplicates of one experiment.

FIG. 3A is a schematic of an experimental timeline of EG.5.1 challenge of five-week-old male Syrian golden hamsters. One day prior to challenge (d−1), the hamsters received an intraperitoneal injection with mAb-52 or 1G05 isotype control at 10 mg/kg. The following day (d0), the hamsters were intranasally challenged with 104 PFU of SARS-CoV-2 EG.5.1. Following challenge, the hamsters were monitored daily, and their nasal wash, nasal turbinate, and lungs were analyzed.

FIG. 3B is a set of graphs showing nasal washes of hamsters challenged with EG.5 and harvested on day 3 for measurements of infectious virus by plaque assay (left) and viral RNA by RT-qPCR (right). The data is from one experiment with 6 hamsters per group/experiment. P values were determined by two-tailed Mann-Whitney test.

FIG. 3C is a set of graphs showing nasal turbinates of hamsters challenged with EG.5 and harvested on day 3 for measurements of infectious virus by plaque assay (left) and viral RNA by RT-qPCR (right). The data is from one experiment with 6 hamsters per group/experiment. P values were determined by two-tailed Mann-Whitney test.

FIG. 3D is a set of graphs showing lungs of hamsters challenged with EG.5 and harvested on day 3 for measurements of infectious virus by plaque assay (left) and viral RNA by RT-qPCR (right). The data is from one experiment with 6 hamsters per group/experiment. P values were determined by two-tailed Mann-Whitney test.

FIG. 4A is an image of XVV.1.5 RBD deep mutational scanning escape of mAb-52. Total escape at each site in the XBB.1.5 receptor binding domain (RBD) as measured by pseudovirus deep mutational scanning library.

FIG. 4B is an image of the escape caused by individual mutations at key sites of escape. XBB.1.5 wild type amino acids are depicted with X and amino acids in grey are absent in the library or highly deleterious for spike function.

FIG. 4C is a graph of KD fold change determined by BLI binding of RBD escape mutants binding Fab-52. RBD mutants with KD fold change >10 are considered the footprint of mAb-52.

FIG. 4D is a set of images of mutational escape at key residues highlighted in heat map on the surface to represent the RBD (left) and the associated ammino acids (right). The higher intensity depicts higher escape. The epitope can be categorized as class I/II based on previous RBD antibody nomenclature.

FIG. 5A is a schematic of 2.58 Å cryo-EM density for Fab-52-XBB.1.5 Spike trimer complex; Fab-52 VH (orange red) and VL (yellow) bound to the RBD (light gray).

FIG. 5B is a schematic of Fab-52 footprint (orange), as defined by buried surface area, depicted on a surface representation of the RBD (light gray) with CDR loops of the Fab-52 VH (orange red) and VL (yellow), ACE2 footprint is outlined as red line track on RBD.

FIG. 5C is a schematic of RBD epitope residues denoted by arrows in FAb-52: RBD interface.

FIG. 5D is a schematic of Fab-52 paratope residues denoted by arrows in orange red and yellow.

FIG. 5E is a schematic of a 180° view along the y-axis (view 1) that show details of the Fab-52: RBD molecular interface with numerous polar interactions.

FIG. 5F is a schematic of a 180° view along the y-axis (view 2) that show details of the Fab-52: RBD molecular interface with numerous polar interactions.

FIG. 6 is a set of flow cytometry plots depicting lymph node fine needle aspirate flow cytometry gating strategy. The representative flow plots analyze TFH, S+ germinal center B cells and lymph node plasma cells.

FIG. 7A is a UMAP depicting transcription clusters from scRNA-seq of an entire lymph node.

FIG. 7B is a dot plot of the marker genes used for annotating the transcriptional clusters in FIG. 7A.

FIG. 7C is a UMAP depicting transcriptional clusters from scRNA-seq of B cells isolated from a lymph node.

FIG. 7D is a dot plot of the marker genes used for annotating the transcriptional clusters in FIG. 7C.

FIG. 7E is a set of UMAPs of B cells found in S+ clones overlayed in red for each participant.

FIG. 7F is a heatmap of the IGHV gene usage by S+ B cell clones with the color intensity corresponding to clone counts.

FIG. 8 contains diagrams summarizing longitudinal tracking of S-binding B cell clones across lymph node and peripheral blood compartments of five subjects. The circos diagrams show clonal overlap between S-binding plasmablasts (PB, red arc), memory B cells (MBC, pink arc), and GC B cells (GCB, blue arc) compartments at indicated time points. Arcs represent B cell compartments, with arc lengths being proportional to the total number of B cells in each compartment. Chords represent clonal connections spanning at least two compartments, with the widths of chord ends corresponding to the number of B cells from the respective compartments. Purple, green and grey chords correspond to clonal connections spanning GCB-PB, GCB-MBC and PB-MBC compartments respectively. Percentages are of B cell clones in the compartment represented by the arc that were related to B cells in indicated compartment and time point.

FIG. 9 is a set of graphs showing authentic SARS-CoV-2 variant neutralizations. Neutralization of mAbs against WA1/2020 D614G, BA.1, XBB.1.5, EG.5.1, BA.2.86, HV.1, JN.1, and KP.2. IC50 values indicated above the graphs. Results are from technical duplicates of one experiment.

FIG. 10A is a set of graphs showing biolayer interferometry (BLI) binding affinity of mAb-52 binding RBD of WA1, BA.1, EG.5.1, HV.1, JN.1, KP.2.

FIG. 10B is a set of graphs showing the BLI binding competition against class I/A-binding mAb 2B04 (top) and ACE2 (bottom). Results are from kinetic measurements of dilution series of one experiment.

FIG. 11A is a set of graphs showing Fab-52 binding affinity against key RBD escape mutants identified in deep mutational scanning.

FIG. 11B is a set of graphs showing Fab-52 binding affinity against key RBD escape mutants identified in deep mutational scanning.

FIG. 11C is a heatmap showing key residue mutational escape in XBB.1.5 RBD library. XBB.1.5 wild type amino acids are depicted with an X mark, and amino acids in grey are absent in the library or highly deleterious.

FIG. 11D is a set of charts showing ELISA binding area under the curve (AUC) of pooled plasma (d28; left) and mAb-52 (right) binding RBD escape mutants. RBD mutants binding mAb-52 with >10% reduction in overall binding were considered epitopic residues of mAb-52. The mutations highlighted in gray box are confirmed escape mutants. Results are from technical duplicates of one experiment.

FIG. 12A is a representative image of an electron micrograph to assess cryo-EM data processing and local resolution.

FIG. 12B is a set of representative images of 2D class average obtained for SARS-CoV-2 XBB.1.5 spike ectodomain in complex with 52 Fab.

FIG. 12C is a map and corresponding graph of local resolution and gold-standard Fourier shell correlation curves generated with cryoSPARC v4.3.1 for the overall complex.

FIG. 12D is a map and corresponding graph of local resolution and gold-standard Fourier shell correlation curves generated with cryoSPARC v4.3.1 for locally refined, RBD-Fab complex.

FIG. 12E is a schematic overview of the cryo-EM data processing workflow including the initial model, NUR 3D classification, NUR CTF refine NUR, and local refinement.

FIG. 13 is a set of images of multiple sequence alignment showing SARS-CoV-2 spike RBD (aa 319-542) of XBB.1.5 (SEQ_ID_NO: 10), WA.1 (SEQ_ID_NO: 11), BA.1 (SEQ_ID_NO: 12), EG.5 (SEQ_ID_NO: 13), BA.2.86 (SEQ_ID_NO: 14), HV.1 (SEQ_ID_NO: 15), JN.1 (SEQ_ID_NO: 16), and KP.2 (SEQ_ID_NO: 17). Epitope residues of Fab-52 are indicated with colored dots, orange red for heavy chain and yellow for light chain.

FIG. 14A is an image ACE2 (PDB-8IOU) buried surface area epitope surface representation.

FIG. 14B is an image mAb-52 buried surface area epitope surface representation.

FIG. 15A is a set of graphs showing BLI binding affinity of mature, germline revertant and germline Fab-52 binding RBD of WA1.

FIG. 15B is a set of graphs showing BLI binding affinity of mature, germline revertant and germline Fab-52 binding RBD of BA.1.

FIG. 15C is a set of graphs showing BLI binding affinity of germline Fab-52 binding variant RBD of WA1, BA.1, EG.5.1, HV.1, JN.1, KP.2. Results are from kinetic measurements of dilution series of one experiment.

Those of skill in the art will understand that the drawings described above are for illustrative purposes only. The drawings are not intended to limit the scope of the present teachings in any way.

### DETAILED DESCRIPTION OF THE INVENTION

The present disclosure is based, at least in part, on the discovery of a human mAb (mAb-52) that is capable of neutralizing all SARS-CoV-2 variants isolated to date. In various aspects, the antibody was cloned from responding B cells isolated from a healthy volunteer after a SARS-CoV-2 bivalent vaccination as described in the Examples below.

In various aspects, the antibody targets the receptor binding domain of the SARS-CoV-2 spike protein and competes with ACE2. Without being limited to any particular theory, these characteristics of the disclosed antibody contribute to its potent neutralization capacity. As used herein, ACE2 refers to angiotensin-converting enzyme 2 that acts as a functional receptor on cell surfaces through which SARS-CoV-2 enters host cells.

As described in the Examples herein, escape mutants against the virus could not be generated in vitro, indicating that the disclosed mAb targets critical and conserved residues in the virus.

In various aspects, the disclosed mAb comprises a broadly protective therapeutic against SARS-CoV-2.

One aspect of the present disclosure provides for compositions comprising mAb-52.

Compositions comprising mAb-52 demonstrated reactivity with a broad range of SARS-CoV-2 variants including XBB1.5, WA.1, BA.1, EG.5, BA.2.86, HV.1, JN.1, AND KP.2.

In one aspect, a cohort of human subjects received mRNA encoding prefusion stabilized WA1/2020 and BA.1 (mRNA-1273, 97.4% S and 93.3% RBD sequence conservation to WA1/2020) SARS-CoV-2 Spike(S) protein, and mAb-52 was identified from a plurality of candidate mAbs using a series of binding assays on Spike RBD domains, as well as virus neutralization assays on a variety of SARS-CoV-2 variants.

In another aspect, hamsters treated with mAb-52 and subsequently challenged with EG.5.1 (104 PFU) variant of SARS-CoV-2 demonstrated lower viral RNA loadings relative to controls.

Disclosed herein are compositions, methods, and treatment plans for treating an individual who is at risk of having a respiratory viral infection, has mild symptoms of a respiratory viral infection, or has severe symptoms of a respiratory viral infection. A composition of the present disclosure may be used to treat, prevent, or reduce the infectivity of a respiratory viral infection. A treatment plan may comprise administering a composition of the disclosure to an individual at risk of having a viral infection or who has a viral infection, thereby preventing or treating the viral infection. In some embodiments, viral transmission may be prevented or reduced by reducing viral infection in the upper respiratory tract. Compositions and methods of the disclosure provide robust antigen-specific antibodies, neutralizing antibodies, and B and T cell responses. This confers protection against infection with marked reductions in viral yield, inflammation, and pathology in the lung. Compositions and methods of the disclosure generate robust mucosal immunity including high levels of neutralizing and anti-RBD IgA and IgG in the serum and lung and SARS-CoV-2 specific resident memory T cells in the lung.

The disclosed compositions and methods protect against SARS-CoV-2 infection in the nasal passages, upper airways, lung tissues, and all other sites of possible dissemination. In various aspects, the disclosed compositions protect against infections by a broad range of SARS-CoV-2 variants including, but not limited to, ancestral WA1/2020 and variants BA.1, XBB.1.5, EG.5.1, BA.2.86, HV.1, and JN.1.

In various aspects, the composition of the present disclosure may be formulated for a variety of administration methods including, but not limited to, intranasally (e.g., as a nasal spray, or inhalation), or systemically (e.g., intravenous or intraperitoneal) and administered for treating or preventing a respiratory viral infection (e.g., a coronavirus infection such as SARS-CoV-2). The compositions of the present disclosure may be administered to a subject who may be at risk of contracting a viral infection (e.g., SARS-CoV-2). For example, the compositions of the present disclosure may be administered to individuals in high-risk environments (e.g., healthcare workers), individuals who have been or who are suspected to have been exposed to a virus (e.g., SARS-CoV-2), or individuals who have tested positive for a viral infection. A composition of the present disclosure may be administered to an individual who is displaying symptoms of a respiratory infection (e.g., a SARS-CoV-2 infection) or who is asymptomatic at the time of administration.

The methods and compositions disclosed herein may be used to treat, prevent, or reduce the infectivity of a respiratory viral infection. In some embodiments, the viral infection may be a coronavirus infection. Pathogens with long incubation periods, such as SARS-CoV-2 which has a median incubation period of about five days, may have a high risk of transmission since many infected individuals may be unaware that they are infected. Additionally, carriers of coronavirus may frequently be asymptomatic or have mild symptoms, leading to unknowing contact between a viral host and other members of a population. A subject at risk for a coronavirus infection may come in contact with an asymptomatic carrier of the coronavirus infection, thereby unknowingly contracting the coronavirus infection. Methods and compositions are needed to prevent coronavirus infections in at-risk individuals (e.g., individuals who have come in contact with a carrier of a coronavirus or who may come in contact with a carrier of a coronavirus). In some embodiments, the compositions, methods, or treatment regiments disclosed herein may treat or prevent a SARS-CoV-2 infection (e.g., COVID-19).

In one example, a desired response is to inhibit or reduce or prevent CoV (such as SARS-CoV-2) infection. The CoV infection does not need to be completely eliminated or reduced or prevented for the method to be effective. For example, administration of an effective amount of the immunogen can induce an immune response that decreases the CoV infection (for example, as measured by infection of cells, or by number or percentage of subjects infected by the CoV) by a desired amount, for example by at least 50%, at least 60%, at least 70%, at least 80%, at least 90%, at least 95%, at least 98%, or even at least 100% (elimination or prevention of detectable CoV infection), as compared to a suitable control.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on coronavirus S ectodomain, such as a SARS-COV S ectodomain. Epitopes can be formed both from contiguous amino acids or noncontiguous amino acids juxtaposed by tertiary folding of a protein.

A prime-boost vaccination is an immunotherapy including the administration of a first immunogenic composition (the primary vaccine) followed by the administration of a second immunogenic composition (the booster vaccine) to a subject to induce an immune response. The priming vaccine and/or the booster vaccine include a vector (such as a viral vector, RNA, or DNA vector) expressing the antigen to which the immune response is directed. The booster vaccine is administered to the subject after the priming vaccine; a suitable time interval between administration of the priming vaccine and the booster vaccine, and examples of such timeframes are disclosed herein. In some embodiments, the priming vaccine, the booster vaccine, or both the primer vaccine and the booster vaccine additionally include an adjuvant. In one non-limiting example, the priming vaccine is a DNA-based vaccine (or other vaccine based on gene delivery), and the booster vaccine is a protein subunit or protein nanoparticle-based vaccine.

### Compositions

A composition of the present disclosure may comprise one or more active agents. In some embodiments, an active agent may be an agent to prevent, treat, or reduce the infectivity of a viral infection. In some embodiments, treating a viral infection may comprise reducing the infectivity and/or transmission of the virus. In some embodiments, preventing a viral infection may comprise reducing the infectivity and/or transmission of the virus. A composition of the present disclosure may comprise an active agent to prevent a viral infection, an active agent to treat a viral infection, an active agent to reduce the infectivity of a viral infection or a combination thereof. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents, or antioxidants.

The present disclosure relates to non-human adenoviral vector compositions and methods of using an immunogenic composition comprising the adenovirus vector and optionally one or more additional active ingredients, a pharmaceutically acceptable carrier, diluent, excipient or adjuvant to treat or prevent a respiratory viral infection. A SARS-CoV-2 antigen or immunogenic portion thereof, or an mAb targeting the SARS-CoV-2 antigen or immunogenic portion, upon infection of a human, stimulate an immune response, and thereby confer immunity to COVID-19 disease.

In various aspects, the disclosed composition comprises the mAb-52 antibody of the present disclosure, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, PATENT-PCT Via EFS Web 45 78289757.2 phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of noneffervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isomorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Nonlimiting examples of suitable lubricants include minerals such as talc or silica; and fats such as vegetable stearin, magnesium stearate, or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug, and cosmetic colors (FD & C), drug and cosmetic colors (D & C), or external drug and cosmetic colors (Ext. D & C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

### Methods

The present disclosure encompasses methods to treat, prevent, or reduce the infectivity or transmission of a virus in a subject in need thereof. In some embodiments, the methods prevent or reduce the infectivity of a viral infection by preventing the internalization of a virus into a cell of the subject or by preventing the internalization of a viral genome into a cell of the subject. In some embodiments, administration of a composition provided herein, for instance those described in Section II, may create an immune response in a subject that disrupts or prevents an interaction between a viral surface protein (e.g., a spike protein or an envelope protein) and a host receptor protein (e.g., an epithelial angiotensin converting enzyme (ACE)). Administering a composition of the disclosure to a subject at risk for a viral infection may reduce the risk of coronavirus infection in the subject.

The disclosed compositions can be administered to a subject to induce an immune response to the corresponding coronavirus spike protein in the subject. In a particular example, the subject is a human. The immune response can be a protective immune response, for example a response that inhibits subsequent infection with the corresponding coronavirus. Elicitation of the immune response can also be used to treat or inhibit infection and illnesses associated with the corresponding coronavirus.

A subject can be selected for treatment that has, or is at risk for, developing infection with the coronavirus corresponding to the S protein in the immunogen, for example because of exposure or the possibility of exposure to the coronavirus. Following administration of a disclosed immunogen, the subject can be monitored for infection or symptoms associated with the coronavirus, or both.

Typical subjects intended for treatment with the therapeutics and methods of the present disclosure include humans, as well as non-human primates and other animals. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional workups to determine environmental, familial, occupational, and other such risk factors that may be associated with the targeted or suspected disease or condition, as well as diagnostic methods, such as various ELISA and other immunoassay methods to detect and/or characterize coronavirus infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct, or coordinate treatment regimen to other treatments.

The administration of a disclosed composition can be for prophylactic or therapeutic purposes. When provided prophylactically, the disclosed therapeutic agents are provided in advance of any symptom, for example, in advance of infection. The prophylactic administration of the disclosed therapeutic agents serves to prevent or ameliorate any subsequent infection. When provided therapeutically, the disclosed therapeutic agents are provided at or after the onset of a symptom of disease or infection, for example, after development of a symptom of infection with the coronavirus corresponding to the S protein in the composition, or after diagnosis with the coronavirus infection. The therapeutic agents can thus be provided prior to the anticipated exposure to the coronavirus so as to attenuate the anticipated severity, duration, or extent of an infection and/or associated disease symptoms, after exposure or suspected exposure to the virus, or after the actual initiation of an infection.

The compositions described herein, are provided to a subject in an amount effective to induce or enhance an immune response against the coronavirus S protein in the subject, preferably a human. The actual dosage of disclosed compositions will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

A composition according to the disclosure can be used in coordinate (or prime-boost) vaccination protocols or combinatorial formulations. In certain embodiments, compositions and coordinate immunization protocols employ separate transgene or formulations, each directed toward eliciting an anti-viral immune response, such as an immune response to coronavirus S proteins. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed transgene. In some examples, the boost may be the same transgene as another boost, or the prime. The prime and boost can be administered as a single dose or multiple doses, for example two doses, three doses, four doses, five doses, six doses or more can be administered to a subject over days, weeks or months. Multiple boosts can also be given, such as one to five (e.g., 1, 2, 3, 4, or 5 boosts), or more. Different dosages can be used in a series of sequential immunizations. For example, a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen, and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, a composition of the disclosure is administered in a single dose.

Upon administration of a disclosed composition of this disclosure, the immune system of the subject typically responds to the composition by producing antibodies specific for the coronavirus S protein included in the composition. Such a response signifies that an immunologically effective dose was delivered to the subject.

In some embodiments, the antibody response of a subject will be determined in the context of evaluating effective dosages/immunization protocols. In most instances, it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum that bind to an antigen including, for example, the recombinant coronavirus S protein included in the immunogen.

Coronavirus infection does not need to be completely eliminated or reduced or prevented for the methods to be effective. For example, elicitation of an immune response to a coronavirus with one or more of the disclosed compositions can reduce or inhibit infection with the coronavirus by a desired amount by at least 10%, at least 20%, at least 50%, at least 60%, at least 70%, at least 80%, at least 90%, at least 95%, at least 98%, or even at least 100% (elimination or prevention of detectable infected cells), as compared to infection with the coronavirus in the absence of the composition. In additional examples, coronavirus replication can be reduced or inhibited by the disclosed methods. Coronavirus replication does not need to be completely eliminated for the method to be effective. For example, the immune response elicited using one or more of the disclosed compositions can reduce replication of the corresponding coronavirus by a desired amount, for example, by at least 10%, at least 20%, at least 50%, at least 60%, at least 70%, at least 80%, at least 90%, at least 95%, at least 98%, or even at least 100% (elimination or prevention of detectable replication of the coronavirus), as compared to replication of the coronavirus in the absence of the immune response.

In some embodiments, the disclosed composition is administered to the subject simultaneously with the administration of the adjuvant. In other embodiments, the disclosed composition is administered to the subject after the administration of the adjuvant and within a sufficient amount of time to induce the immune response.

In some embodiments, the administration of a therapeutically effective amount of one or more of the disclosed compositions to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art and are further described herein, and include, but are not limited to, plaque reduction neutralization (PRNT) assays, microneutralization assays, flow cytometry-based assays, single-cycle infection assays. In some embodiments, the serum neutralization activity can be assayed using a panel of coronavirus pseudoviruses. For example, to test the immunogenicity of the vaccine candidates against multiple MERS-COV strains-without the requirement of a biosafety level 3 facility—a pseudotyped reporter virus neutralization assay was previously developed (Wand et al., Nat Commun, 6:7712, 2015), similar to that previously developed for SARS-COV (Martin et al, Vaccine 26, 6338, 2008; Yang et al, Nature 428, 561, 2004; Naldini et al, PNAS 93, 11382, 1996; Yang et al, PNAS 102, 797, 2005).

In other embodiments, the present disclosure provides methods to treat, prevent, or reduce the infectivity of a respiratory viral infection. In some embodiments, the viral infection may be a coronavirus infection. The coronavirus may be SARS-COV, SARS-CoV-2, MERS-COV, HKU1, OC43, or 229E. The coronavirus may be a beta-coronavirus. A subject at risk for a coronavirus infection may come in contact with an asymptomatic carrier of the coronavirus infection, thereby unknowingly contracting the coronavirus infection.

The methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas, and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6 (log 10 [Na+])+0.41 (fraction G/C content)−0.63 (% formamide)−(600/I). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by any number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-IT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, SARS-CoV-2 infections can be modulated (e.g., reduced, eliminated, or enhanced) using genome editing. Processes for genome editing are well known; see e.g. Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of SARS-CoV-2 infections by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)20NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for SARS-CoV-2 infections.

For example, the methods described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing SARS-CoV-2 infections in a subject in need of administration of a therapeutically effective amount of a mAb-52 composition, so as to protect against SARS-CoV-2 infections.

Methods described herein are generally performed on a subject in need thereof. A subject in need of the therapeutic methods described herein can be a subject having, diagnosed with, suspected of having, or at risk for developing a SARS-CoV-2 infection. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an mAb-52 is, for example, an amount that would cause the desired therapeutic effect in a subject while minimizing undesired side effects. In various embodiments, an effective amount of an mAb-52 described herein can substantially inhibit SARS-CoV-2 infections, slow the progress of SARS-CoV-2 infections, or limit the development of SARS-CoV-2 infections. In some aspects, a therapeutically effective amount of mAb-52 may range from 5 mg/kg to 15 mg/kg. In some aspects, a therapeutically effective amount of mAb-52 may be 10 mg/kg.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a mAb-52 composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to protect against SARS-CoV-2 infections.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a mAb-52 composition can occur as a single event or over a time course of treatment. For example, a mAb-52 composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for SARS-CoV-2 infections.

A mAb-52 composition can be administered simultaneously or sequentially with another agent, such as an antibiotic, an anti-inflammatory, or another agent. For example, a mAb-52 composition can be administered simultaneously with another agent, such as an antibiotic or an anti-inflammatory. Simultaneous administration can occur through the administration of separate compositions, each containing one or more of a mAb-52 composition, an antibiotic, an anti-inflammatory, or another agent. Simultaneous administration can occur through the administration of one composition containing two or more of a mAb-52 composition, an antibiotic, an anti-inflammatory, or another agent. A mAb-52 composition can be administered sequentially with an antibiotic, an anti-inflammatory, or another agent. For example, a mAb-52 composition can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about-2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to, an mAb-52 antibody, antibiotics, solubilizing agents, salts, nanoparticle compositions. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Defining a Highly Conserved B Cell Epitope in the Receptor Binding Motif of SARS-CoV-2 Spike Glycoprotein

In this example, studies were preformed to identify, isolate, and evaluation the mAb-52 is disclosed herein.

SARS-CoV-2 mRNA vaccines induce robust and persistent germinal center (GC) B cell responses in humans. It remains unclear how the continuous evolution of the virus impacts the breadth of the induced GC B cell response. Using ultrasound-guided fine needle aspiration, we examined draining lymph nodes of nine healthy adults following bivalent booster immunization. We show that 77.8% of the B cell clones in the GC expressed as representative monoclonal antibodies recognized the spike protein, with a third (37.8%) of these targeting the receptor binding domain (RBD). Strikingly, only one RBD-targeting mAb, mAb-52, neutralized all tested SARS-CoV-2 strains, including the recent KP.2 variant. mAb-52 utilizes the IGHV3-66 public clonotype, protects hamsters challenged against the EG.5.1 variant and targets the class I/II RBD epitope, closely mimicking the binding footprint of ACE2. Finally, we show that the remarkable breadth of mAb-52 is due to the somatic hypermutations accumulated within vaccine-induced GC reaction.

### Introduction

The continuous evolution of severe acute respiratory syndrome coronavirus 2 (SARS-CoV-2) has led to the emergence of viral variants of concern and a reduction in the effectiveness of spike(S) protein-derived mRNA vaccines. Multiple studies have demonstrated a reduction in neutralization potency of variants of concern by sera from vaccinees following immunization with a two dose primary series (ancestral WA1/2020) and subsequent boosters. This result has necessitated the annual update of variant derived vaccines to combat the rise in infections. Thus, it is imperative to understand whether these variant vaccines primarily induce recall immune responses to the ancestral virus or predominantly de novo responses specific to the variants of the vaccine formulation to gauge their effectiveness. Several studies have highlighted the recall responses to ancestral WA1/2020 S protein following variant-derived booster S protein mRNA vaccination. We have previously shown that variant-derived bivalent mRNA-1273.213 (B.1.351/B.1.617.2) or monovalent mRNA-1273.529 (Omicron, BA.1) vaccination in humans predominantly elicited recall memory B cell responses in peripheral blood, although limited de novo memory B cell responses targeting novel epitopes can be detected.

Vaccination studies predominantly focus on monitoring immune responses in peripheral blood. However, using ultrasound-guided fine needle aspirations (FNAs), it is possible to sample germinal center (GC) responses in the draining lymph nodes (LNs). The GC reaction is responsible for the selection and affinity maturation of antigen-specific B cell clones, making it critical to understand whether variant-derived vaccines favor the recruitment of memory B cells or naïve clones to induced GCs. Previous studies from our laboratory and others have demonstrated that SARS-CoV-2 mRNA vaccines elicit antibodies targeting three domains: the N-terminal domain (NTD), receptor binding domain (RBD), and S2 domain of the S protein. The majority of neutralizing antibody responses target the RBD. RBD-binding antibodies are subclassified into five groups (class I-V) based on their epitope, with class I/II antibodies targeting the receptor binding motif (RBM), neutralizing the virus by blocking its binding to the host receptor, angiotensin-converting enzyme 2 (ACE2). Antibodies utilizing germline heavy chain genes IGHV3-53/3-66 and targeting class I/II site have been identified as public clonotypes. Eliciting antibody responses incorporating such clonotypes by vaccination will likely not only result in effective neutralization but also create a strong selection pressure for population-level escape.

To investigate the impact of SARS-CoV-2 variant booster vaccines on B cell clonal dynamics, GC recruitment and RBD-binding breadth, we enrolled nine healthy participants who had previously received three doses of mRNA-1273 (100 μg)/BNT162b2 (30 μg) into an observational study. Recruited participants received a single (4th) dose of 50 μg bivalent mRNA-1273.214 (WA1:BA.1=1:1) booster vaccination encoding ancestral and variant-derived S proteins. We used peripheral blood and lymph node FNA samples to assess the degree to which bivalent booster vaccines induced recall or de novo B cell responses.

### Results

B Cell Responses to mRNA-1273.214 Bivalent Booster Vaccination

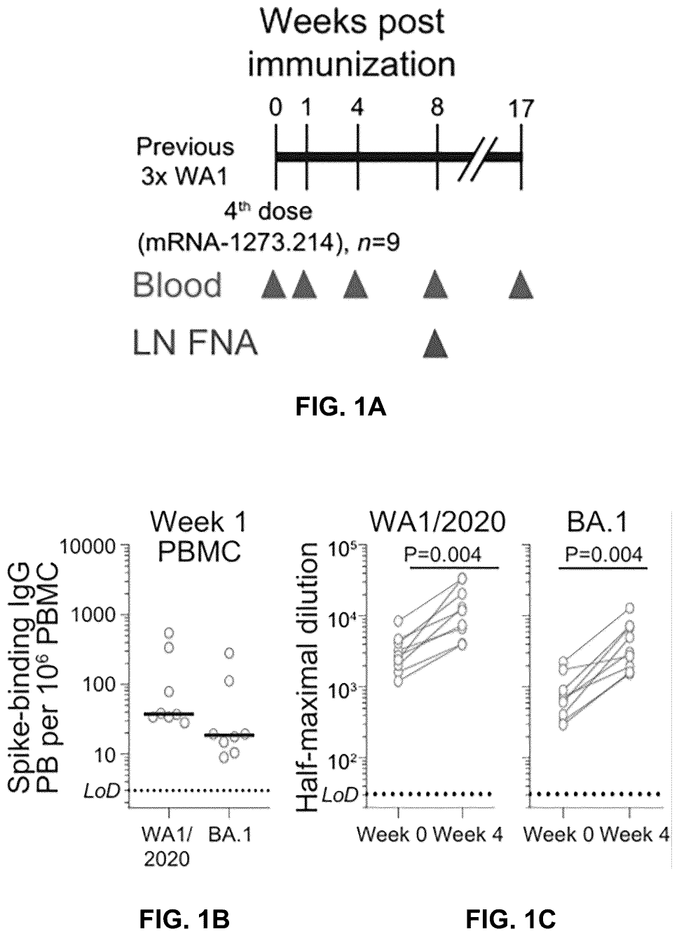

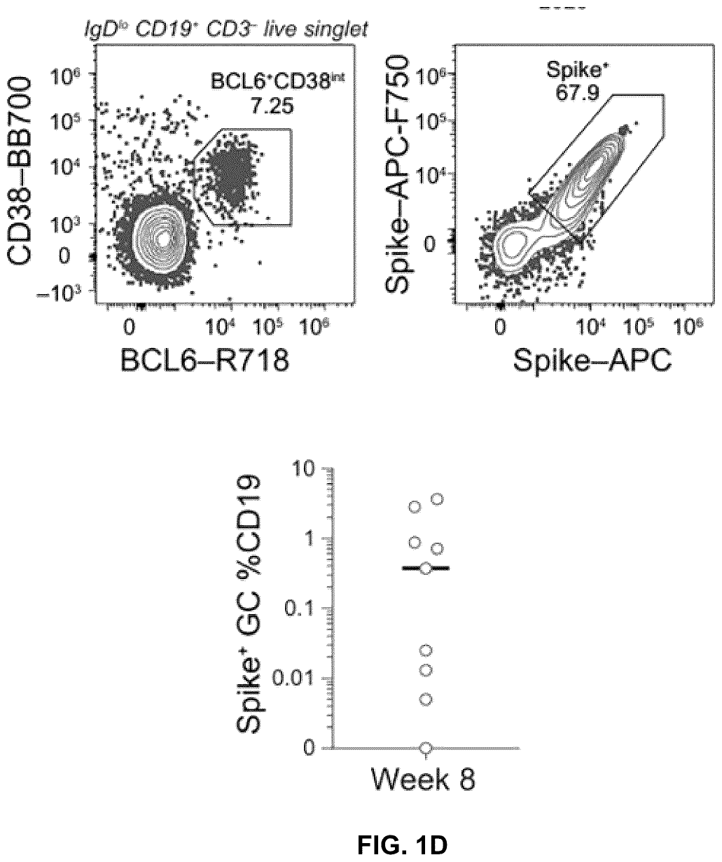

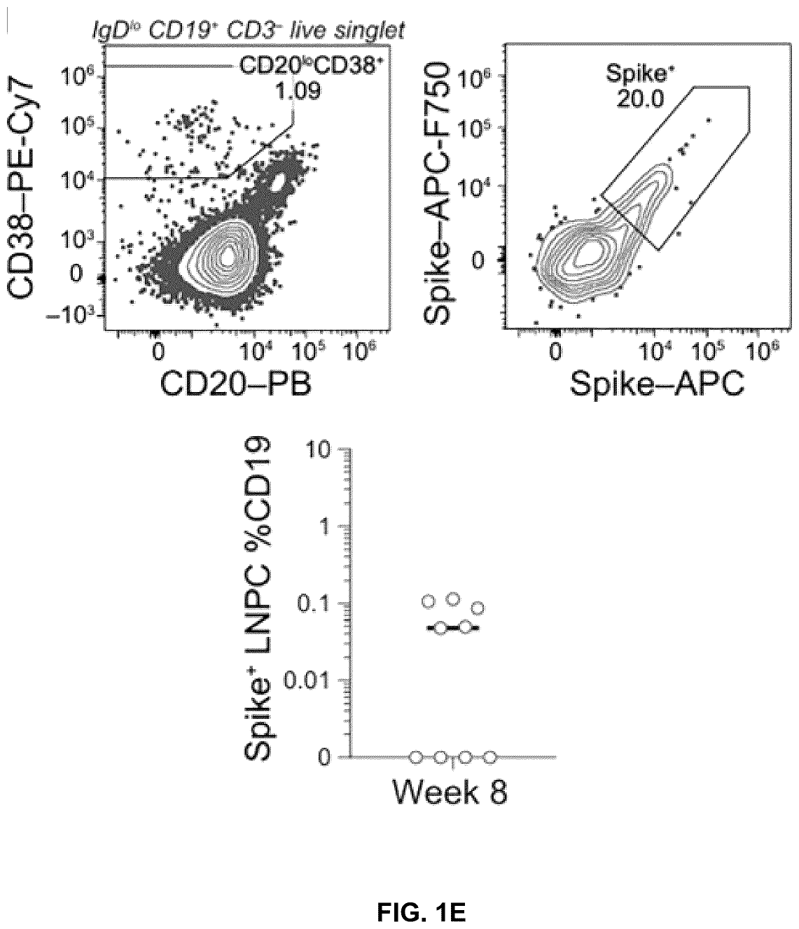

Nine participants were recruited to study WU382 in the spring of 2022. After having received three prior mRNA-1273 or BNT162b2 immunizations targeting the WA1/2020 S protein, individuals were boosted with 50 μg of mRNA (1273.214) encoding prefusion stabilized WA1/2020 and BA.1 (97.4% S protein and 93.3% RBD sequence conservation to WA1) S proteins (Table 1). Blood samples were collected at baseline and at weeks 1, 4, 8, and 17 post boosting, and FNAs of draining axillary LNs were collected at the 8-week time point (FIG. 1A). S⁺ plasmablast (PB) responses were measured in peripheral blood by enzyme-linked immunosorbent spot (ELISpot) assay. We detected robust WA1 and BA.1 S⁺ PB responses one week post boosting in all immunized participants except 382-69, who did not provide sample at day 8 (WA1: 28-547, BA.1:8-280 S⁺ IgG PBs per million PBMCs) (FIG. 1B). The plasma antibody titers increased 2 to 14-fold (Geometric mean titer (GMT): 3.8-fold) against WA1/2020 and 1.5 to 12.3-fold (GMT: 5.3-fold) against BA.1 by week 4 post boosting (FIG. 1C). Eight weeks post boosting, draining lateral axillary LNs were sampled by ultrasound-guided FNAs. WA1 and BA.1 S⁺ GC B cells (CD19⁺CD3⁻IgD^{lo}Bcl6⁺CD38^{int}) were detected in 5 of 9 participants at >0.1% of CD19⁺ cells (FIG. 1D).

TABLE 1
Study participant demographics

| Variable | Total n = 9 n (%) | |
|---|---|---|
| Age (median [range]) | 51 | (27-72) |
| Sex | | |
| Female | 5 | (55.6) |
| Male | 4 | (44.4) |

Bivalent Boosting Recruits Extensively Cross-Neutralizing Clones into Germinal Centers

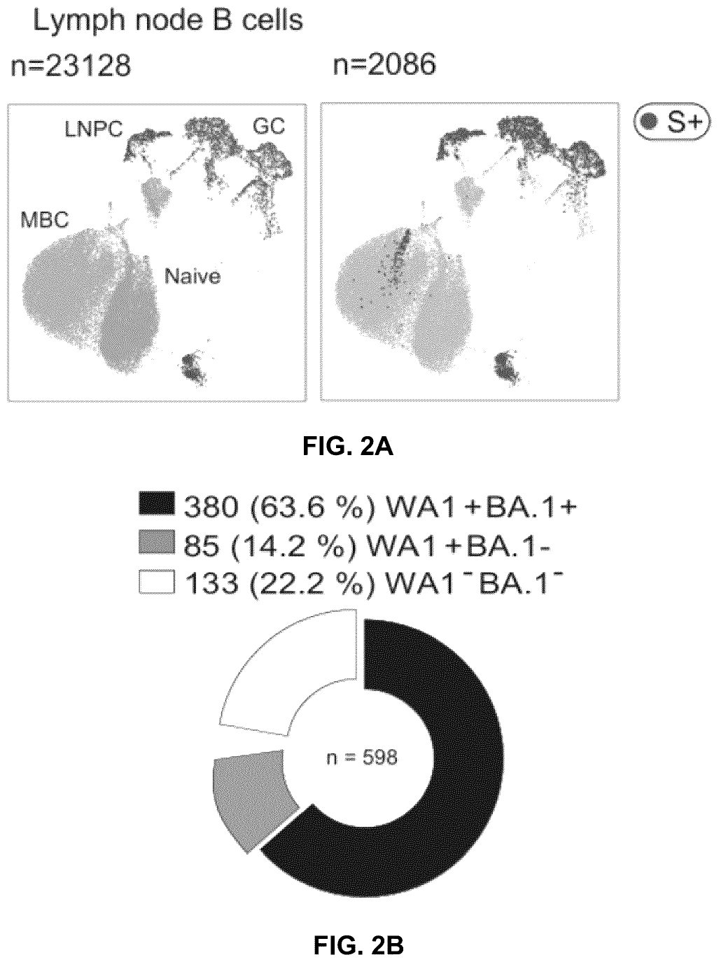

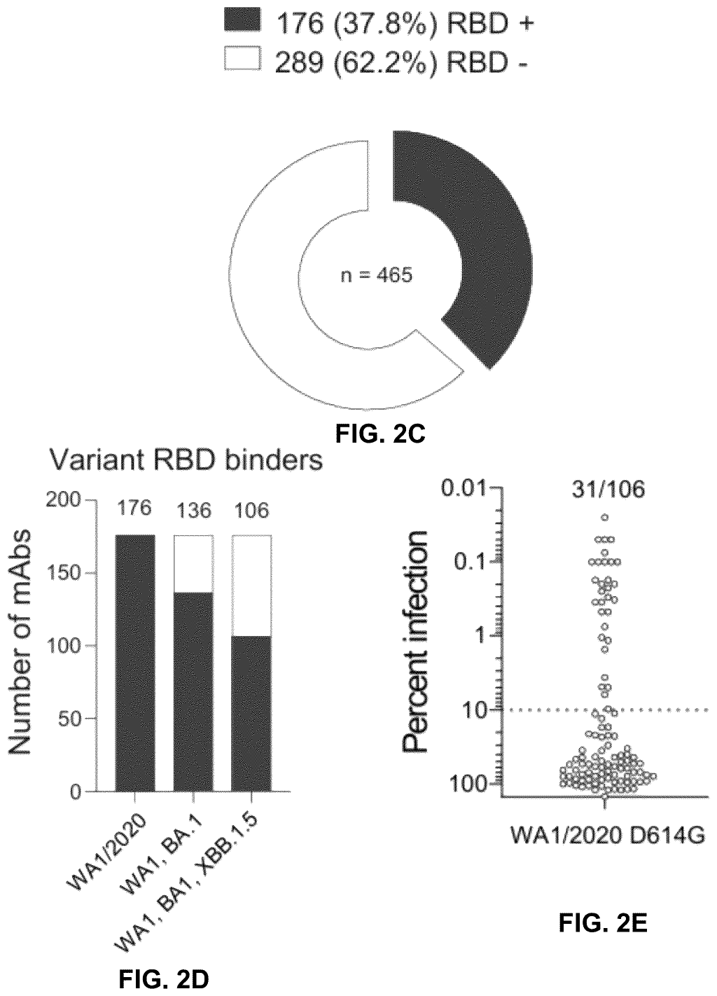

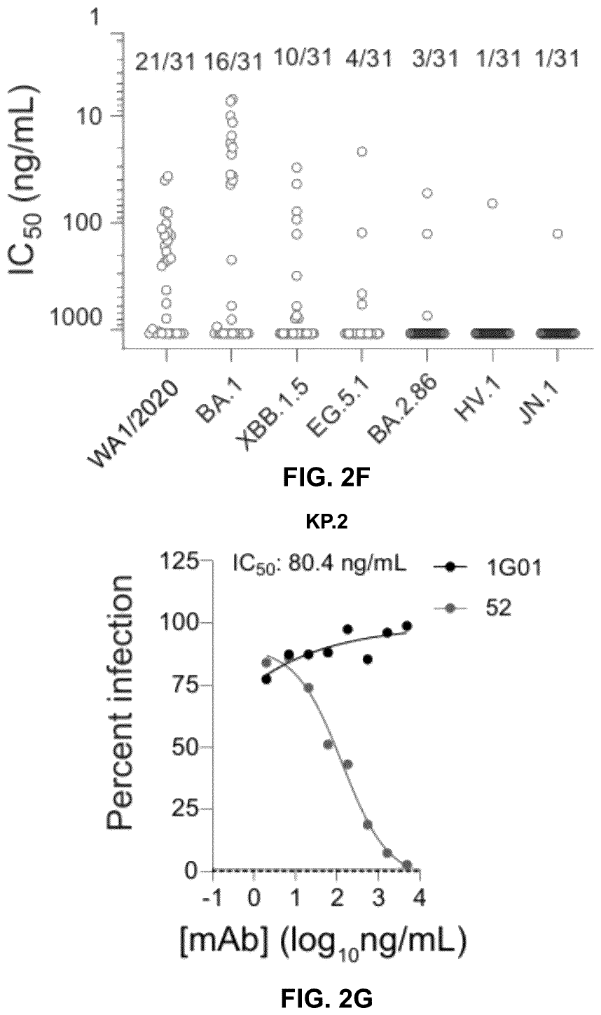

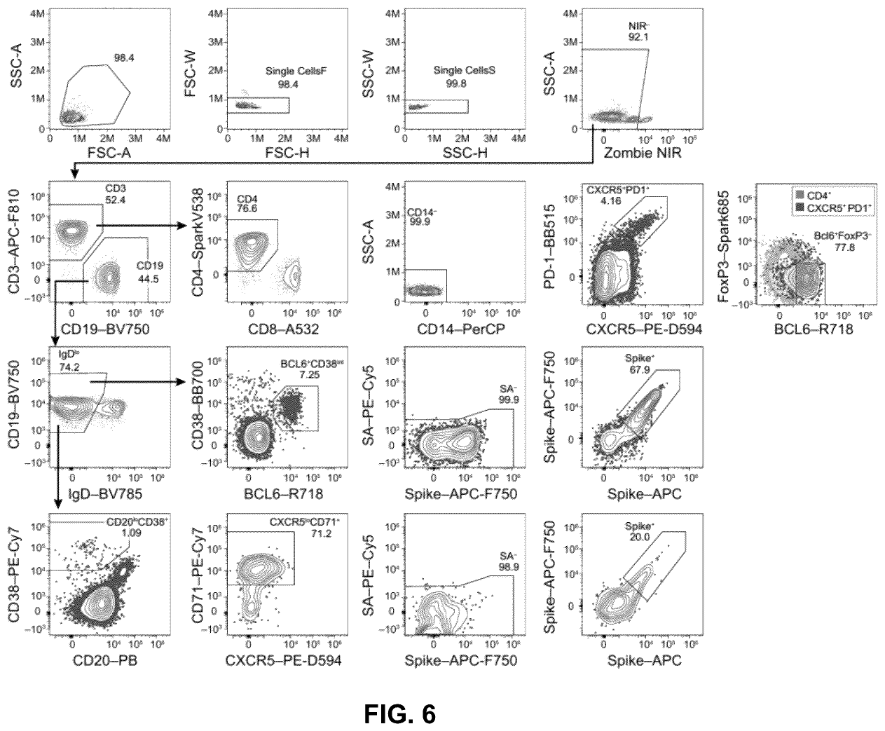

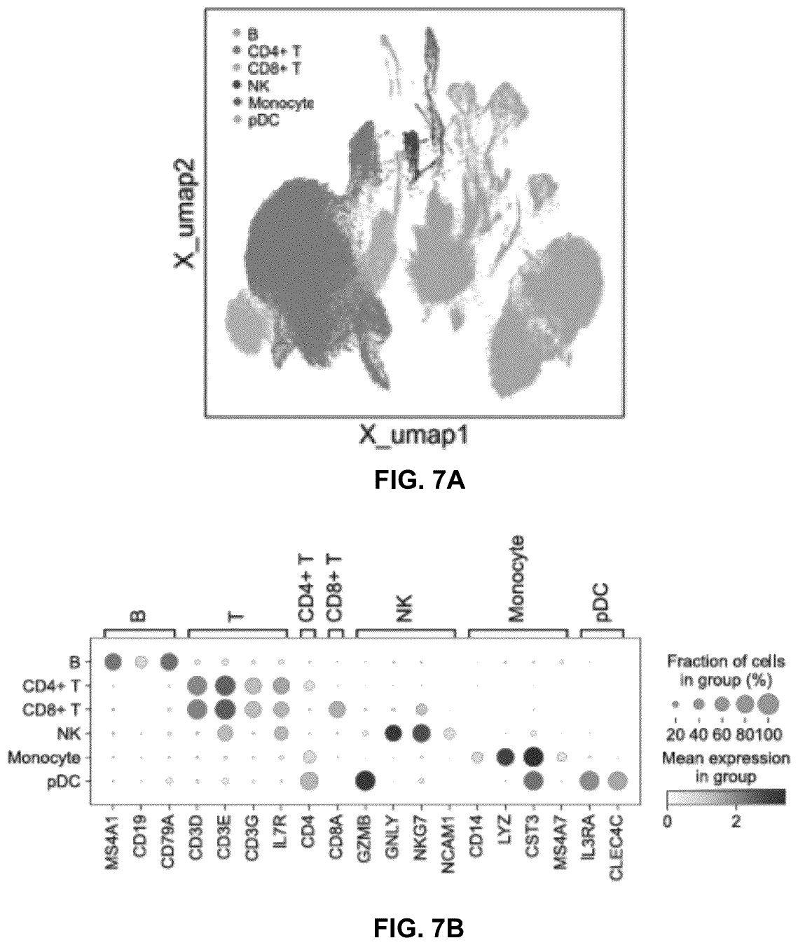

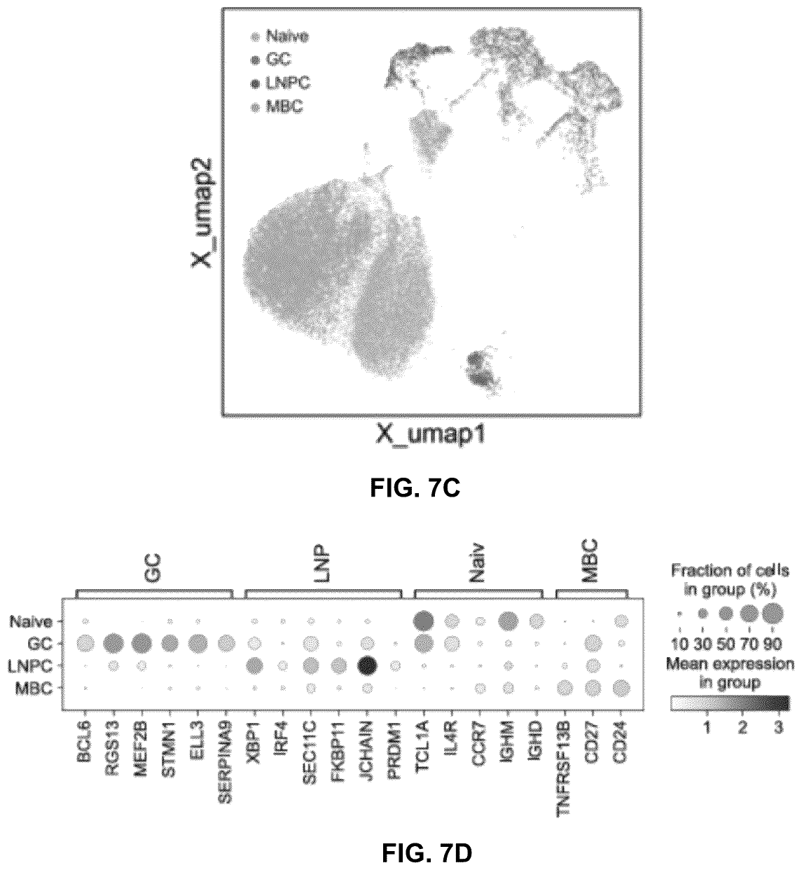

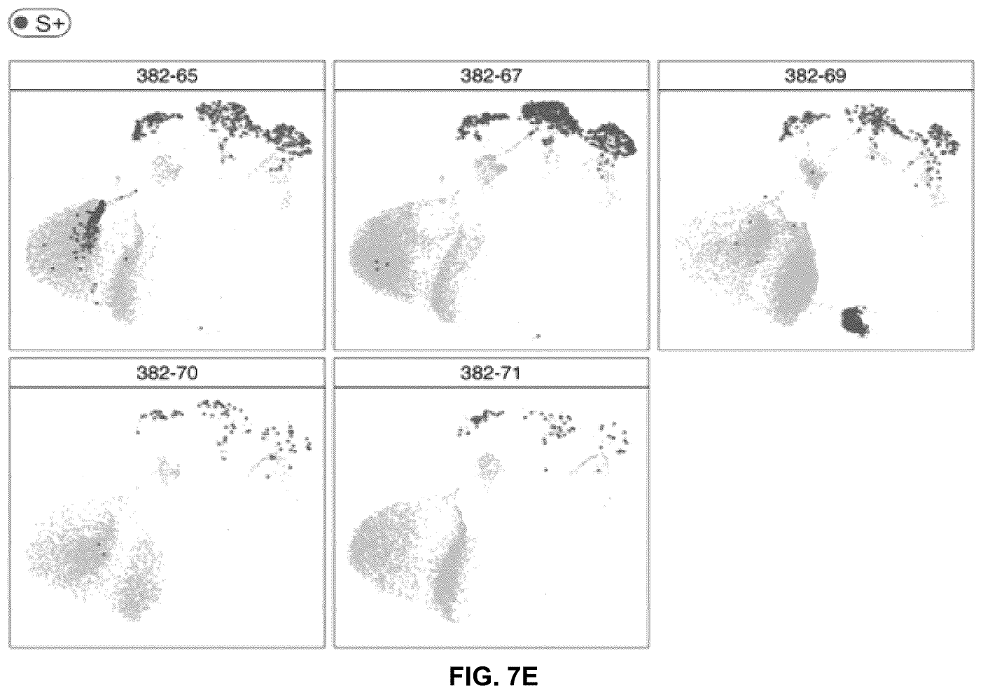

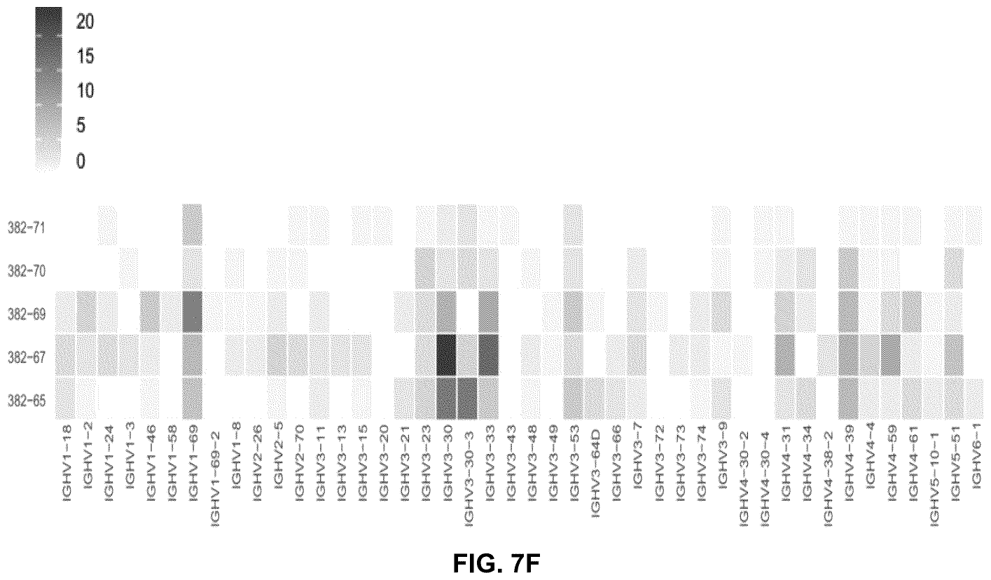

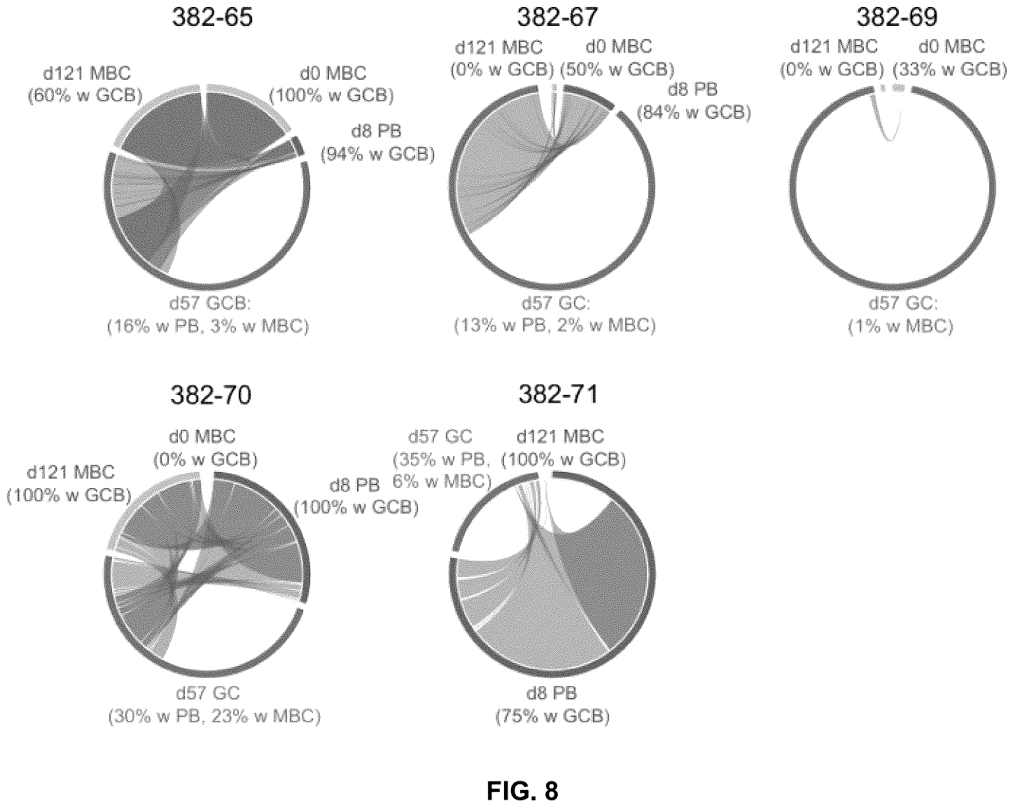

The FNA samples from the 5 participants with detectable S⁺ GC B cells (382-65/67/69/70/71) were selected for single cell RNA sequencing (scRNA-seq) to track clonal dynamics and determine the antigen specificity of B cell clones (FIG. 1D, FIG. 1E, FIG. 6, FIG. 7C). Based on their gene expression profile, scRNA-seq revealed 6 major immune cell clusters typical of a secondary lymphoid organ including B cells, CD4⁺ T cells, CD8⁺ T cells, natural killer cells, monocytes, and plasmacytoid dendritic cells (FIG. 7A, FIG. 7B). Further clustering of B cells (n=23,128) produced four major subclusters: naïve B cells, germinal center (GC) B cells, lymph node plasma cells (LNPC) and memory B cells (MBC) (FIG. 2A, FIG. 7C, FIG. 7D). Using paired heavy and light chain B cell receptor (BCR) sequencing data, we computationally recovered 598 clonally distinct GC B cell and LNPC clones for monoclonal antibody (mAb) generation. We characterized the S protein binding of these mAbs by enzyme-linked immunosorbent assay (ELISA) and mapped the S⁺ mAbs to B cell clones consisting of 2086 single cells (n=2086) across multiple B cell subclusters (FIG. 2A, right). A major fraction (n=465, 77.8%) of the mAbs were WA1 St, while the remaining 22.2% (n=133) clones were non-S-binders (FIG. 2B). The entirety of the St-binders bound WA1, suggesting the GC response was due to recall of MBCs previously exposed to the ancestral S antigen (FIG. 2A, FIG. 2B, FIG. 7E). Further, we longitudinally tracked S-binding GC B cell clones in peripheral blood at baseline (MBC), week 1 (PB), and week 17 (MBC). Clonal tracking revealed consistently high percentages of clonal overlap of GC B cells to PB at week 1, and less consistently so of GC B cells to MBC at baseline, further confirming that the GC B cell response was recall-derived (FIG. 8). We further determined if the S-binders specifically targeted the major neutralizing domain, the RBD. Of the S-specific mAbs, 37.8% (n=176) bound ancestral WA1-derived RBD (RBD⁺), while the remaining were non-RBD (RBD⁻) binders (FIG. 2C).

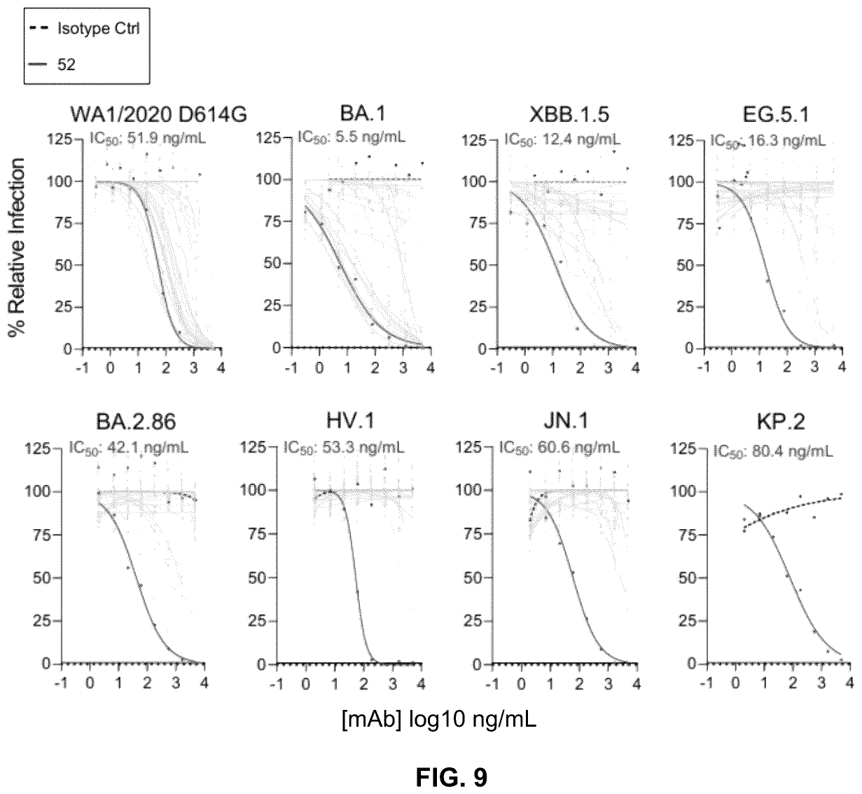

We next characterized the cross reactivity of the clones that bound WA1 RBD (n=176). A majority (60.2%) of the WA1 RBD-binding mAbs cross reacted with RBDs from both BA.1 and XBB.1.5, a subsequent variant of concern (n=106) (FIG. 2D). A minority (29.2%) of these cross reactive mAbs neutralized chimeric vesicular stomatitis viruses (VSV) expressing the WA1/2020 D614G S protein (n=31, >90% inhibition) in a single endpoint neutralization (10 μg/ml) (FIG. 2E). We further assessed the neutralization capacity of these mAbs using an authentic virus neutralization assay against WA1, BA.1, XBB.1.5, EG.5.1, BA.2.86, HV.1, and JN.1 variants (FIG. 2F). We observed a gradual decrease in the number of mAbs that retained inhibitory activity as the antigenic distance of the virus increased from the ancestral strain: WA1 (n=21); BA.1 (n=16); XBB.1.5 (n=10); EG.5.1 (n=4); BA.2.86 (n=3); HV.1 (n=1); and JN.1 (n=1) (FIG. 2F). A minority (n=10) of the mAbs that neutralized VSV-WA1/2020 D614G S at high concentration lost the ability to inhibit infection of authentic WA1/2020 virus, possibly due to differences in the virus neutralization assays, concentration of tested mAbs (10 μg/ml vs 5 μg/ml), or relative expression of S protein on the virion. Notably, we observed a single mAb, mAb-52, which potently neutralized all tested viral strains: ancestral WA1 (IC₅₀: 51.9 ng/ml) and variants BA.1 (IC₅₀: 5.5 ng/ml), XBB.1.5 (IC₅₀: 12.4 ng/ml), EG.5.1 (IC₅₀: 16.2 ng/ml), BA.2.86 (IC₅₀: 42.1 ng/ml), HV.1 (IC₅₀: 53.3 ng/ml), JN.1 (IC₅₀: 60.6 ng/ml), and KP.2 (IC₅₀: 80.4 ng/ml) (FIG. 2F, FIG. 2G, FIG. 9) These results highlight the recruitment of some broadly cross-reactive germinal center B cell clones following bivalent booster vaccination.

GC B Cell-Derived mAb-52 Protects Hamsters from EG.5.1 Challenge

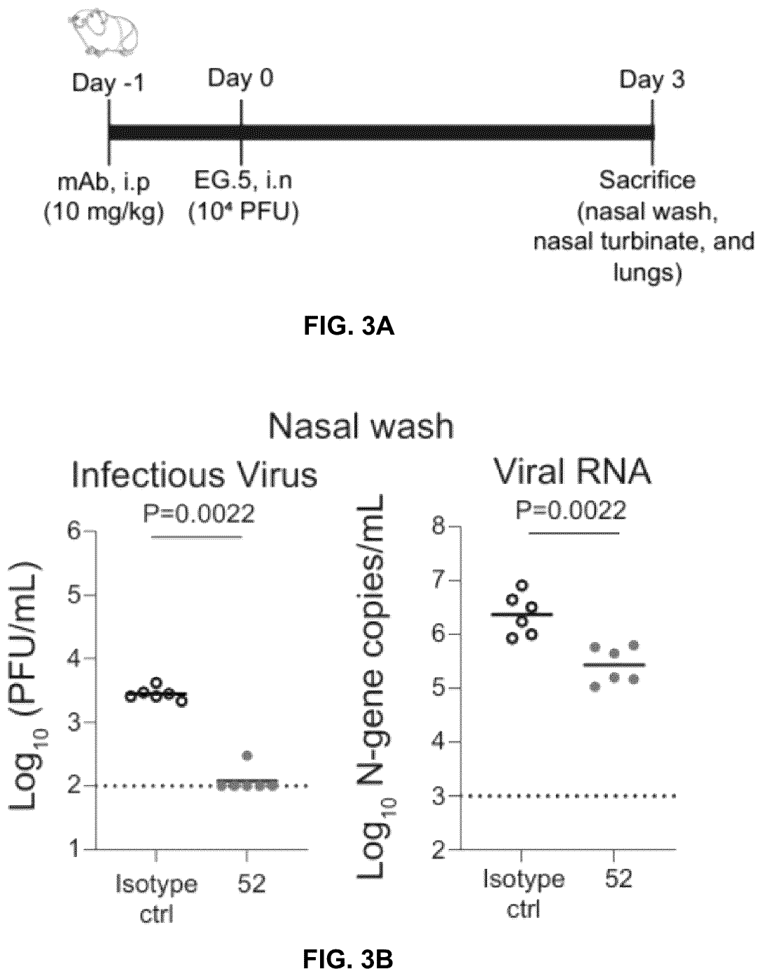

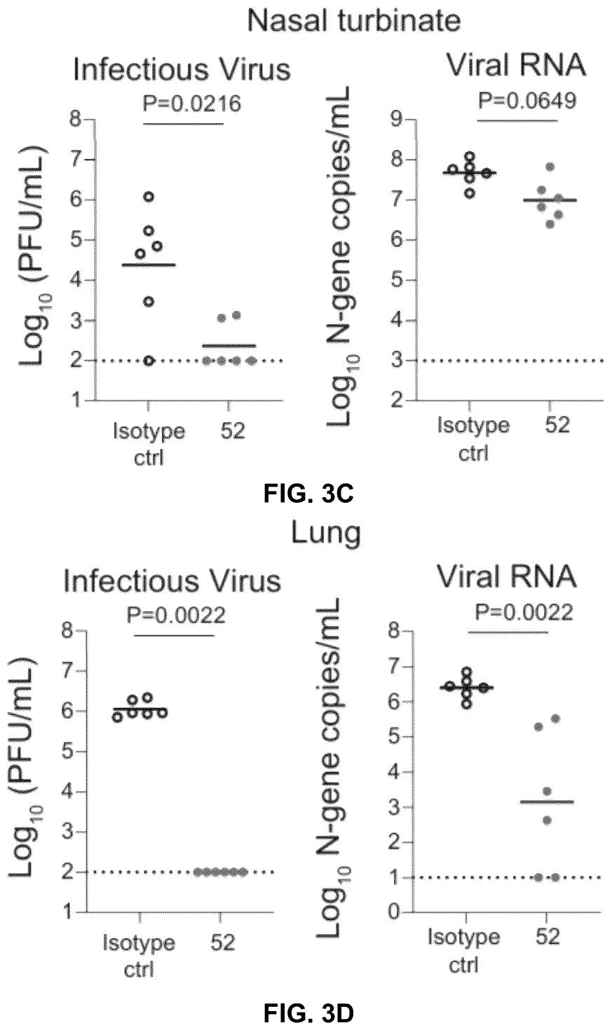

Given the breadth of mAb-52 and possible treatment implications, we next examined its protective efficacy against the EG.5.1 SARS-CoV-2 variant in a hamster challenge model. Six hamsters were treated with mAb-52 or isotype control antibody 1G05 (dose: 10 mg/kg) one day prior to intranasal challenge with the EG.5.1 (104 PFU) variant of SARS-CoV-2 (FIG. 3A). Following challenge, the hamsters were monitored for three days prior to measurement of infectious virus and viral RNA in the nasal wash, nasal turbinate, and left lung homogenates (FIG. 3A). mAb-52-treated hamsters had lower levels of infectious virus in the upper and lower respiratory tract compared to isotype control-treated hamsters (nasal wash: 23-fold, nasal turbinate: 103-fold, lungs: 11, 195-fold) (FIG. 3B, FIG. 3C, FIG. 3D; left). Similarly, we observed lower levels of viral RNA in mAb-52-treated hamsters than the isotype control-treated animals (nasal wash: 8.6-fold, nasal turbinate: 4.7-fold, lungs: 1,804-fold) (FIG. 3B, FIG. 3C, FIG. 3D; right).

mAb-52 Belongs to Public Clonotype IGHV3-66*02 and Targets the Class I/II RBD Epitope

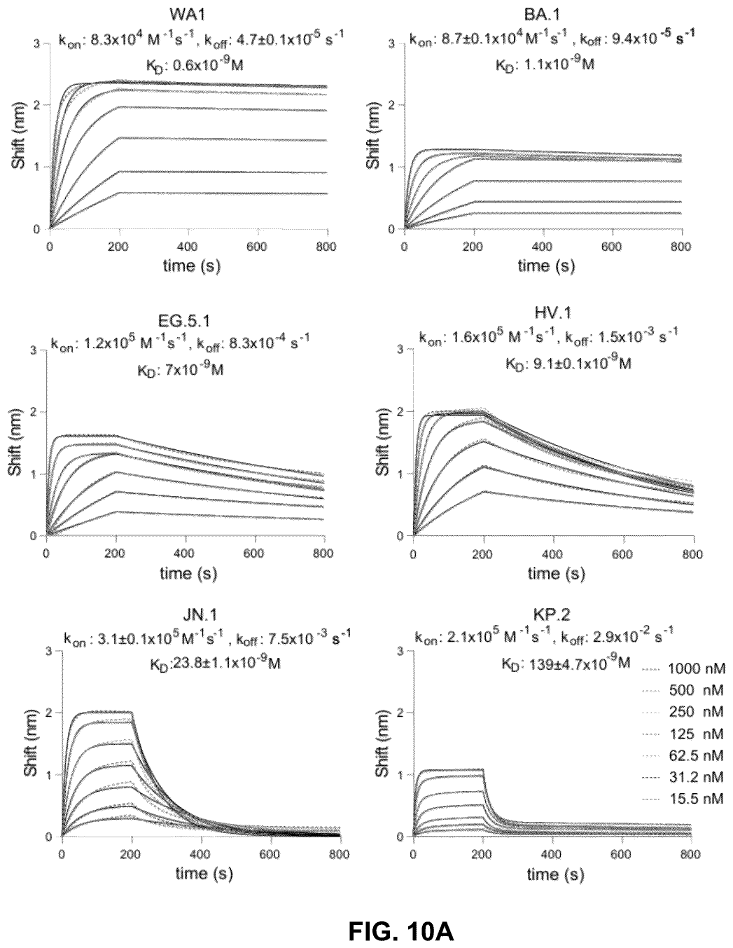

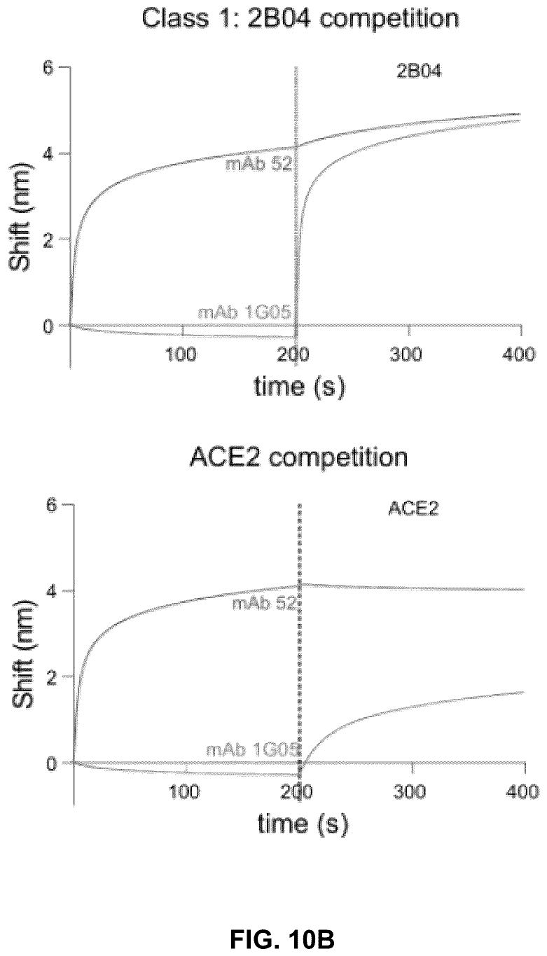

We observed several public B cell clonotypes expressed in the FNA of vaccinated individuals comprising IGHV1-69/3-23/3-30/3-33/4-39/5-51 (FIG. 7F). mAb-52 is encoded by a frequently utilized public clonotype, IGHV3-66*02. Antibodies utilizing germline heavy chain gene IGHV3-66 and binding the RBD often target class I/II epitopes on the RBD. Consistent with prior observations, mAb-52 bound with nanomolar affinity to all RBD variants tested: WA1 (K_{D}=0.6 nM), BA.1 (K_{D}=1.1 nM), EG.5.1 (K_{D}=7 nM), HV.1 (K_{D}=9.1 nM), JN.1 (K_{D}=23.8 nM), and KP.2 (K_{D}=139 nM) (FIG. 10A). We note that the binding affinity is decreasing against newer variants, even though it remains high. mAb-52 competed with ACE2 for binding the RBD, as well as with a previously characterized class I/A-binding monoclonal antibody (2B04), suggesting mAb-52 might engage an epitope similar to or near the ACE2 receptor binding site (FIG. 10B).

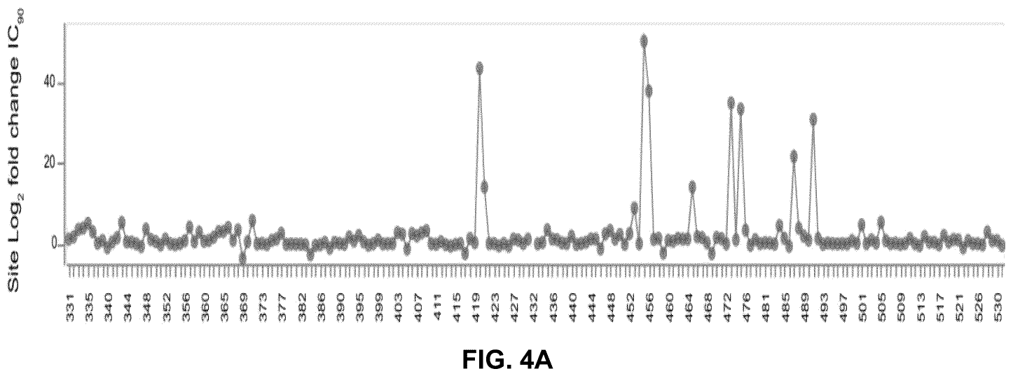

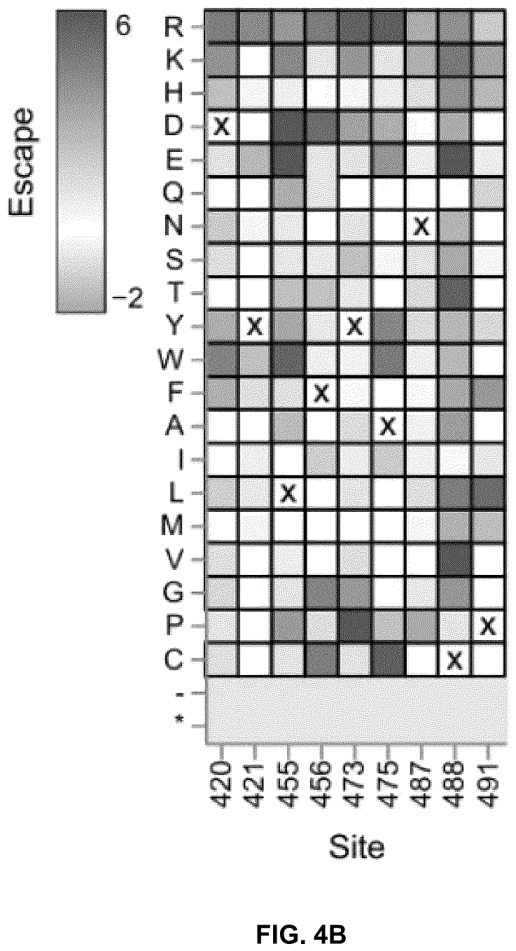

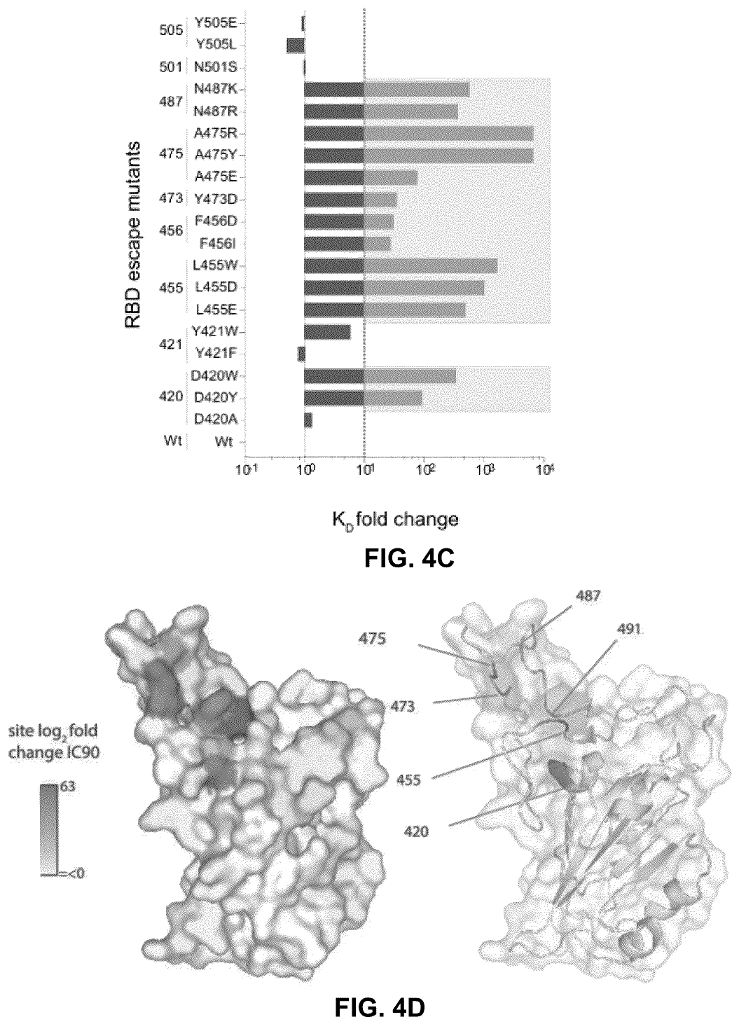

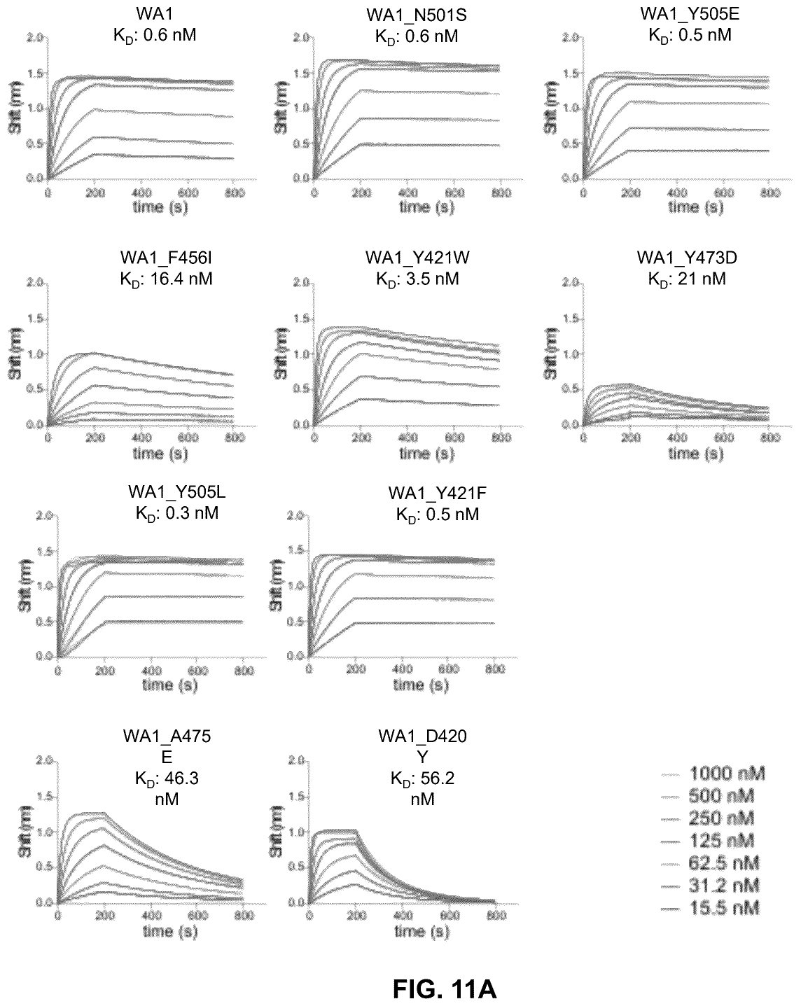

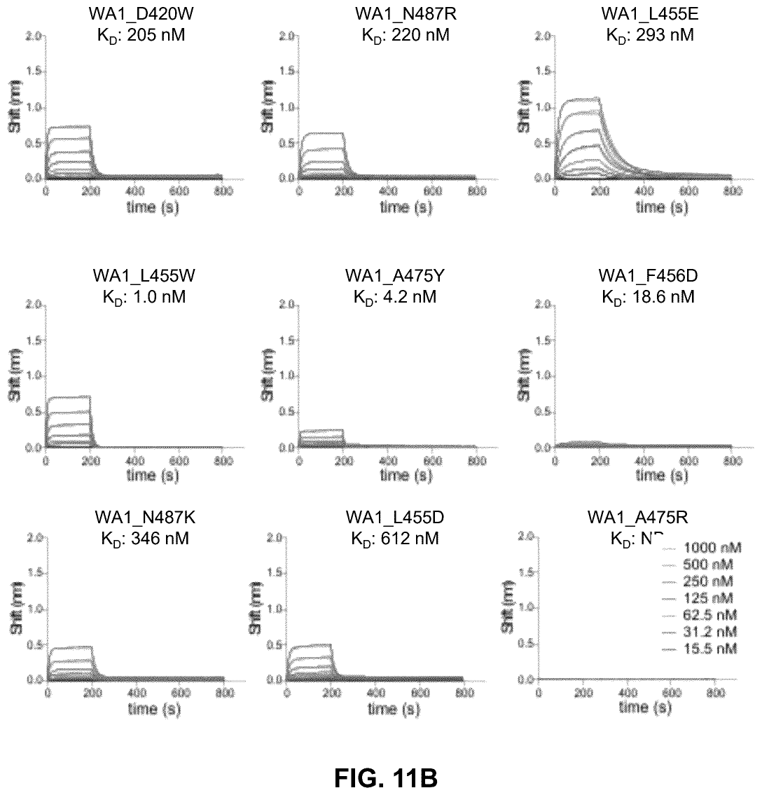

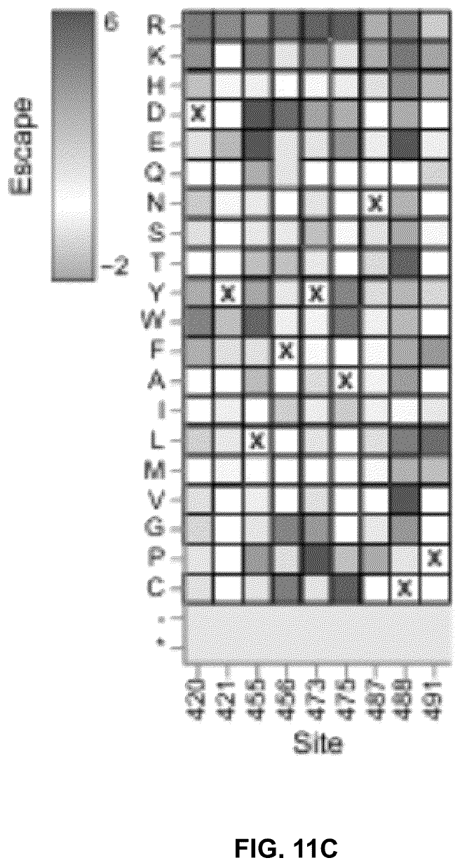

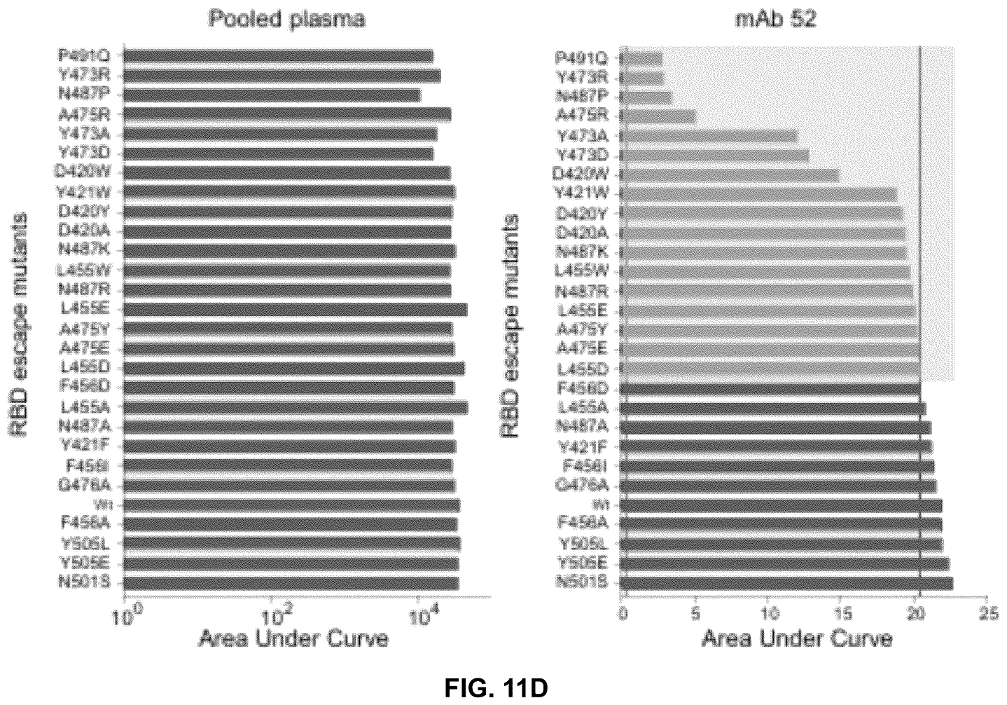

We employed pseudovirus deep mutational scanning (DMS) using libraries of the XBB.1.5 spike with saturating RBD mutations to identify the key sites where mutations escape neutralization by mAb-52. Escape from mAb-52 is caused primarily by mutations at nine spatially clustered sites (420, 421, 455, 456, 473, 475, 487, 488, and 491) (FIG. 4A). At many of these sites, some amino-acid mutations cause more escape than others (FIG. 4B). For instance, at site L455, mutations to charged or large amino acids (e.g., L455D, L455E and L455W) caused strong escape; but mutations to some other amino acids had only a modest effect, likely explaining why mAb-52 still neutralized JN.1 (which contains L455S). We validated key escape mutants by two independent in vitro assay platforms: ELISA testing mAb-52, and biolayer interferometry (BLI) testing the fragment antigen binding (Fab) of mAb-52 (FIG. 4C, FIG. 11A, FIG. 11B, FIG. 11C, FIG. 11D). For these validation experiments, escape mutants were incorporated in the background of ancestral RBD as majority of the escape mutant positions were conserved across XBB.1.5 and WA1/2020. ELISA and BLI assays corroborated the loss of binding, with escape residues losing binding (>10-percent decrease in ELISA AUC or >10-fold increase in BLI K_{D}) to mAb-52 and Fab-52, respectively (FIG. 4C, FIG. 4D, FIG. 11A, FIG. 11B, FIG. 11C, FIG. 11D). Key escape sites determined by DMS for which we validated the effects of mutations in these in vitro assays included residues 420, 421, 455, 456, 473, 475, 487 and 491 (FIG. 4C, FIG. 4D).

Cryo-EM Structure of mAb-52 Complexed with XBB.1.5 Spike

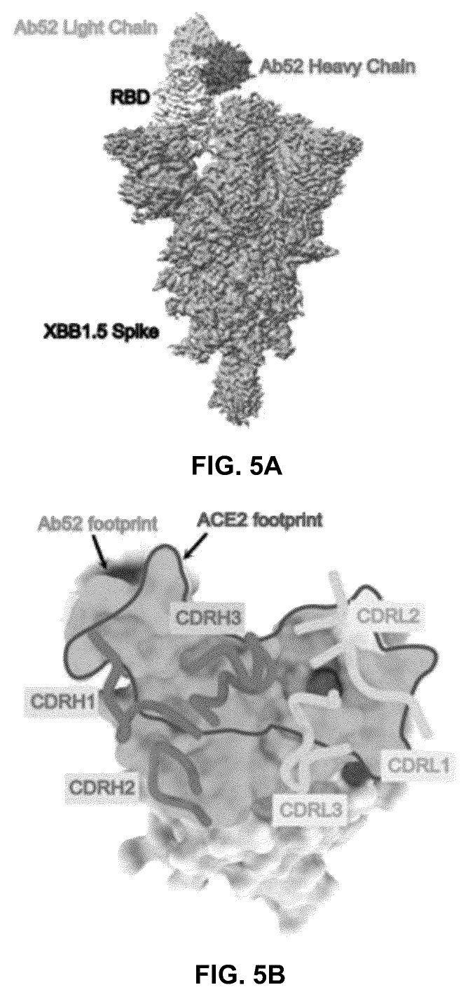

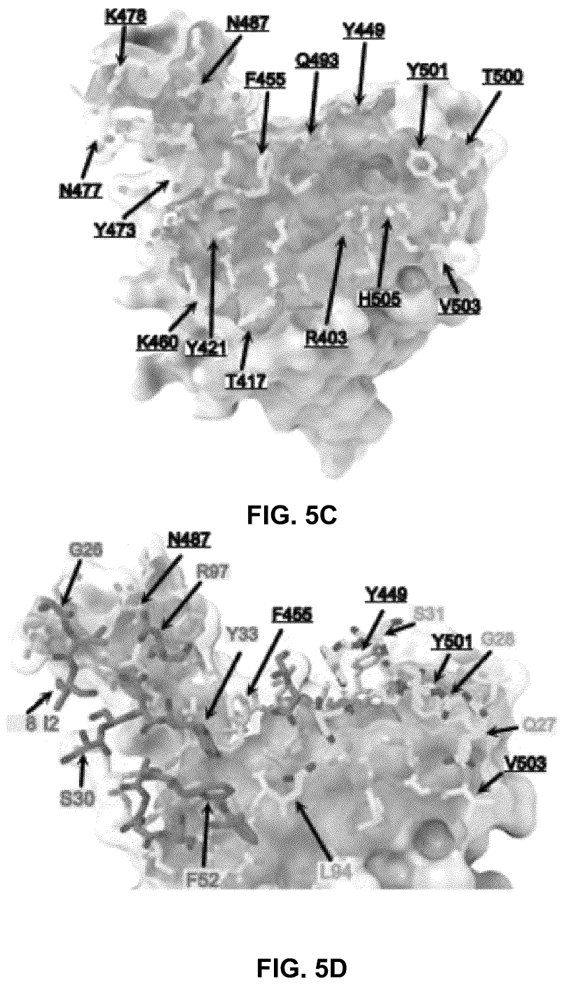

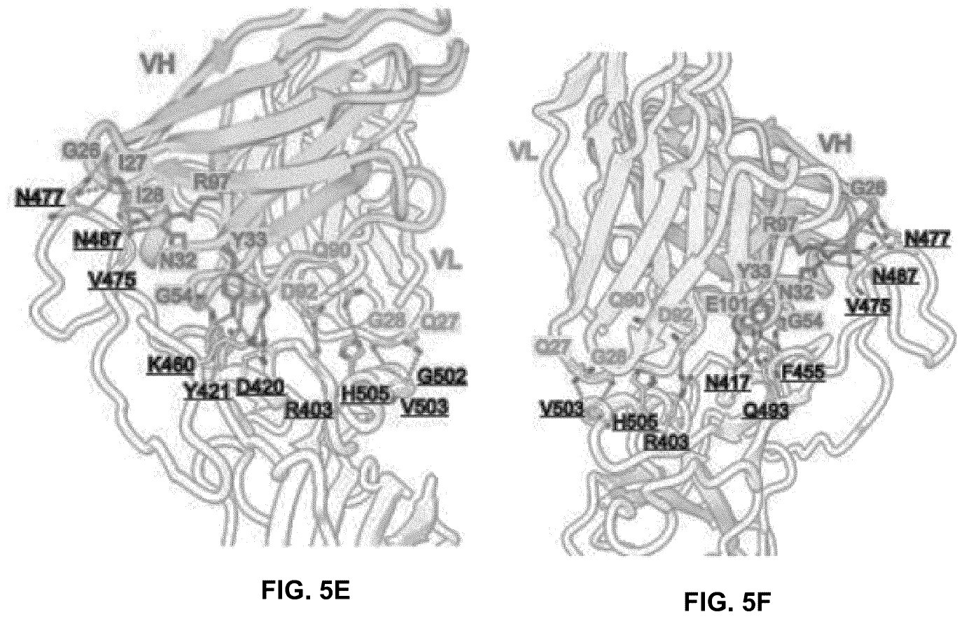

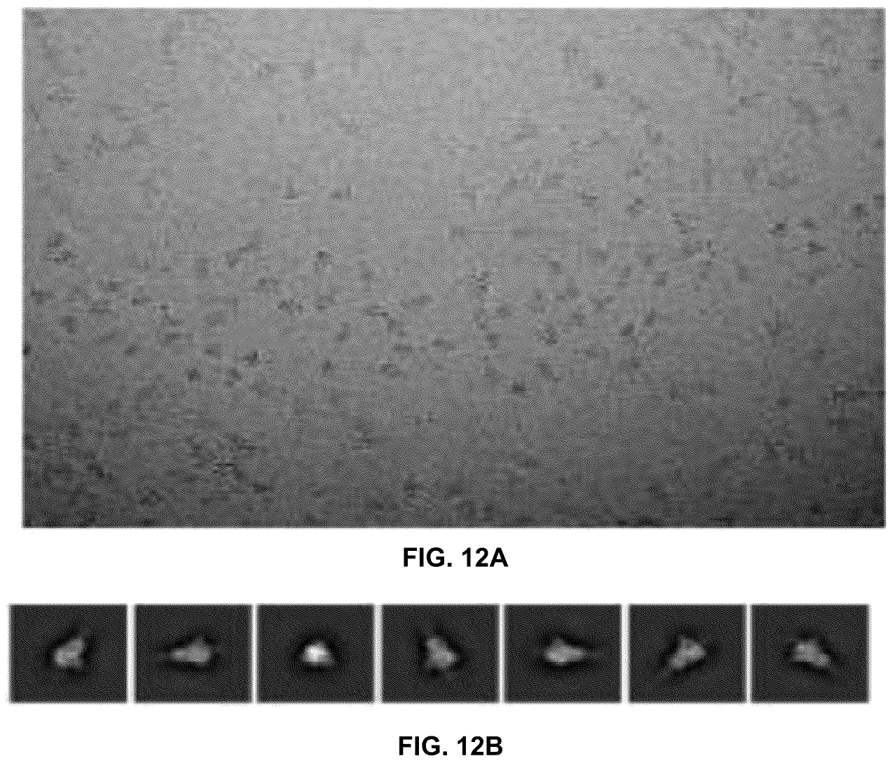

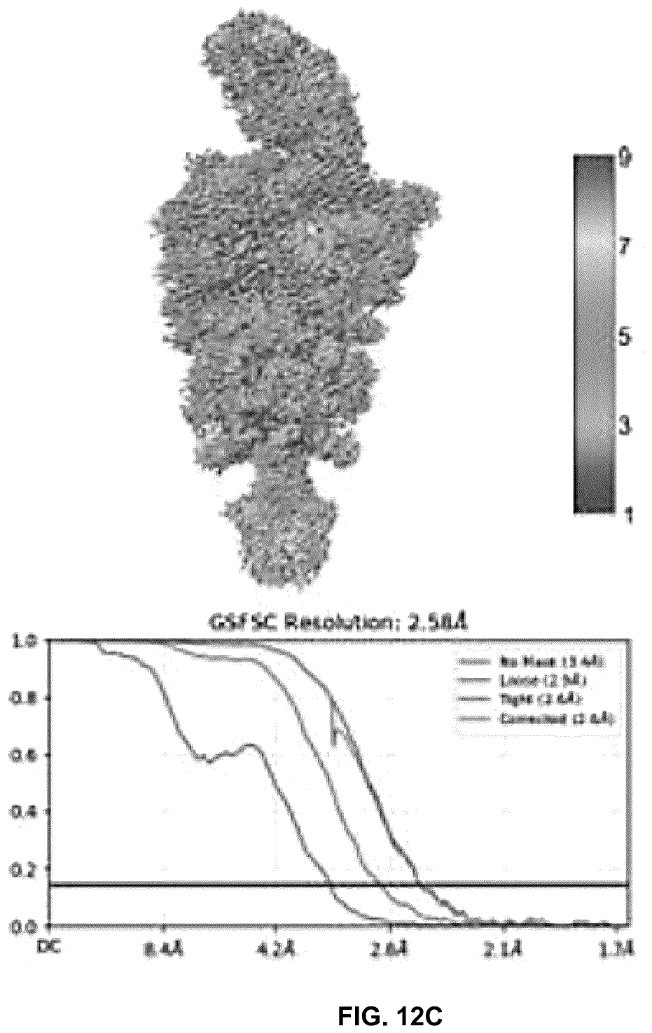

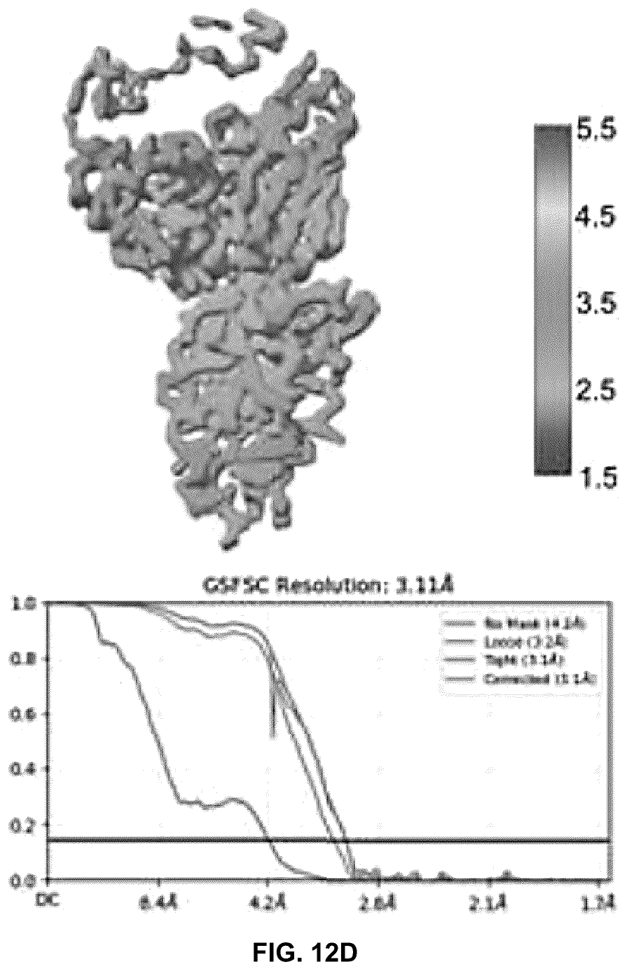

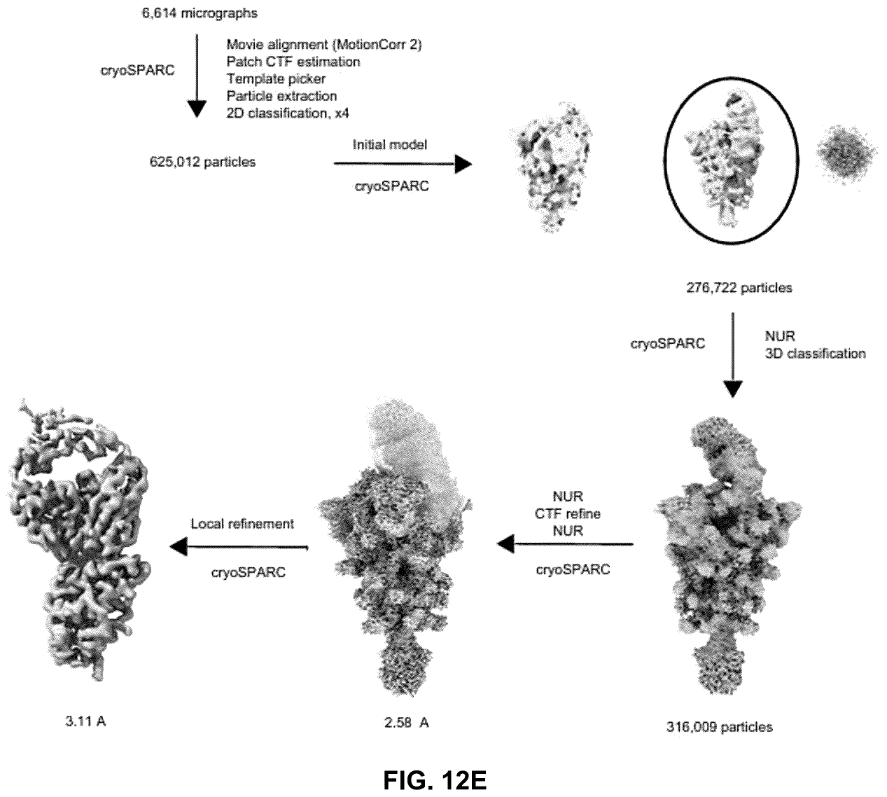

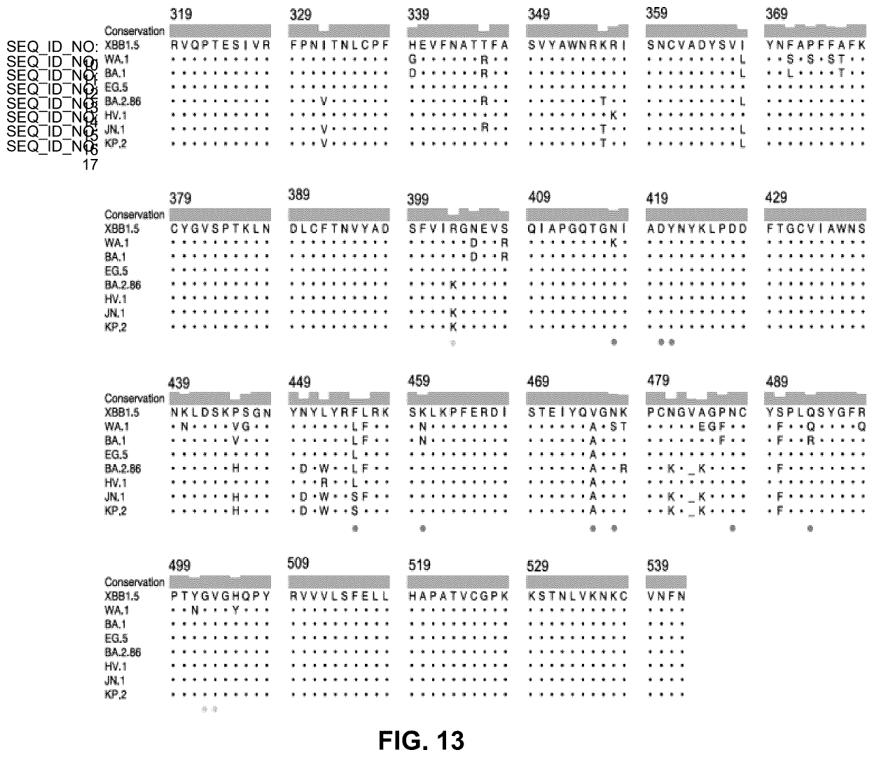

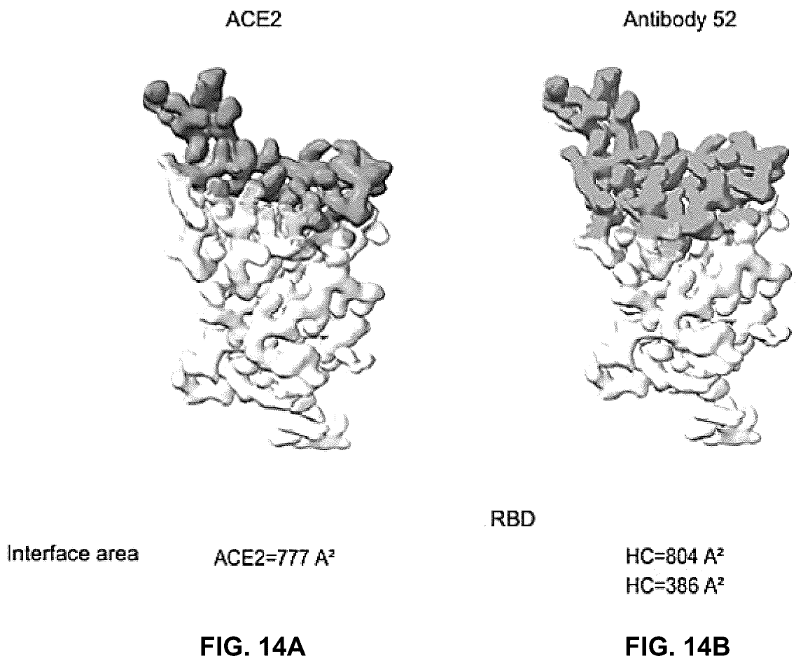

To gain greater molecular insight into the binding epitope targeted by mAb-52, we determined the structure of XBB.1.5 S protein in complex with Fab-52 by cryo-electron microscopy (cryo-EM), yielding a global resolution map at 2.6 Å (FIG. 5A, FIG. 5B, FIG. 12A, FIG. 12B, FIG. 12C, FIG. 12D, FIG. 12E). The XBB.1.5 S used for cryo-EM contains FLip mutations in the RBD (L455F and F456L). Fab-52 bound to one RBD in up-conformation on S protein trimer (FIG. 5A). We improved the antibody: antigen interface resolution by local refinement to achieve a map at 3.1 Å of nominal resolution (FIG. 5B, FIG. 12E). The cryo-EM structure confirmed that mAb-52 targets the class I/II RBD epitope, with the key binding residues in the receptor binding groove composed of F455, K460, Y473, V475, N477, N487, Q493 and the receptor binding motif (RBM) lateral residues D420, Y421 (FIG. 5C, FIG. 5D, FIG. 13). The Fab heavy chain bound the residues in the RBM and the lateral surface, which were otherwise occluded on the RBD in the down conformation. mAb-52 targets several secondary structural regions of the RBD, including alpha helices α4 and α5, and beta sheets 35 and 36 in the RBM, and alpha helix 3 in the periphery of the RBM. The light chain provides supporting interactions with residues R403, G502, V503, and H505 (FIG. 5E, FIG. 5F). The approach angle of mAb-52 binding XBB.1.5 RBD is consistent with previously reported RBD-binding mAbs utilizing the IGHV3-66*02 public clonotype, including CS23, PDI37, C98C7, and others. There are several antibodies characterized with similar HC:LC pairing as mAb-52, but none with determined structures. mAb-52 complexed with S protein cumulatively buried a surface area of 1190 Å² with a higher contribution from the heavy chain (804 Å2) and lower contribution from the light chain (386 Å2) (FIG. 14A, FIG. 14B). Fab-52 engages RBD via polar and hydrophobic interactions involving CDRH1-H3, CDRL1 and CDRL3. mAb-52 residue Y33 of the CDRH1 formed H-bonding interactions with F455 backbone carbonyl oxygen (FIG. 5E, FIG. 5F). G54 and S56 within the CDRH2 formed extensive H-bonding interactions with terminal carbonyl oxygen atoms of Y421 and D420, respectively. Additionally, R97 and E101 of the CDRH3 formed H-bonding with RBD residues N487 and Q493 respectively. Notably, of the 15 hydrogen bonds between Fab-52 and the RBD, six involve the RBD backbone rather than the amino acid side chains (specifically, residues F455, V475, N477, G502 and V503), which allow mAb-52 to maintain its effectiveness despite mutations in the virus spike glycoprotein.

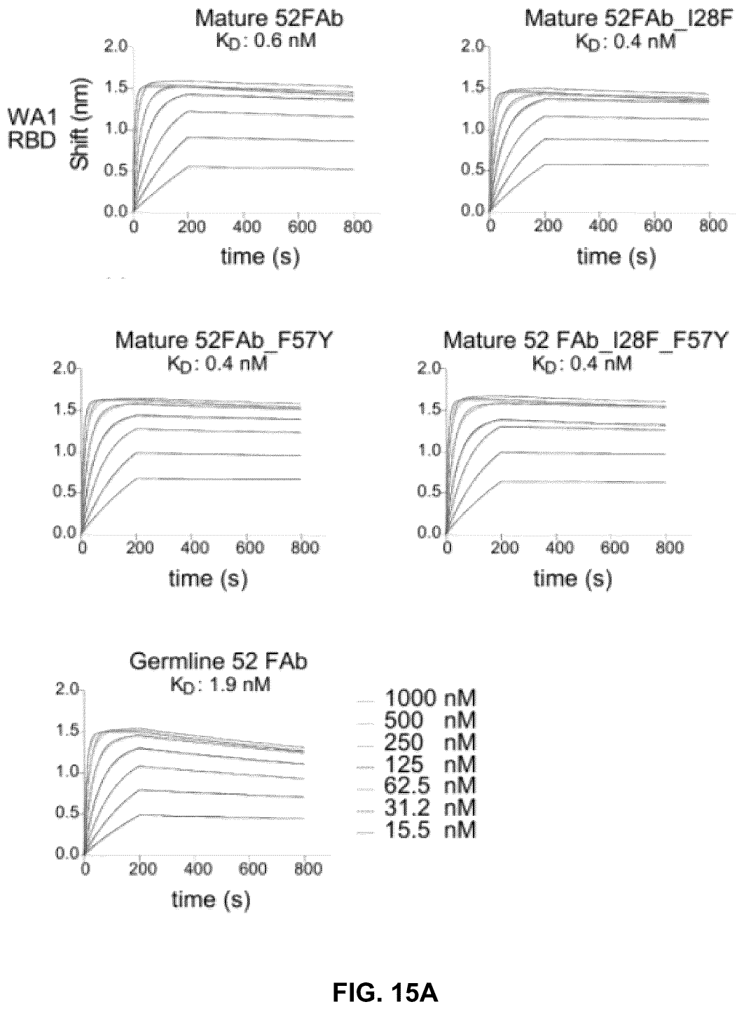

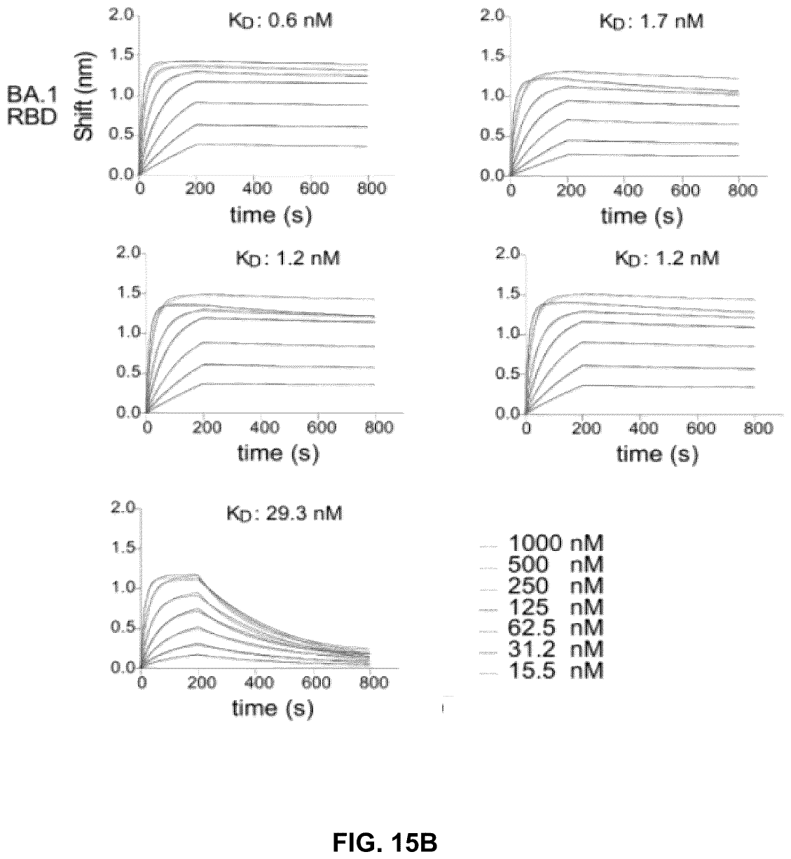

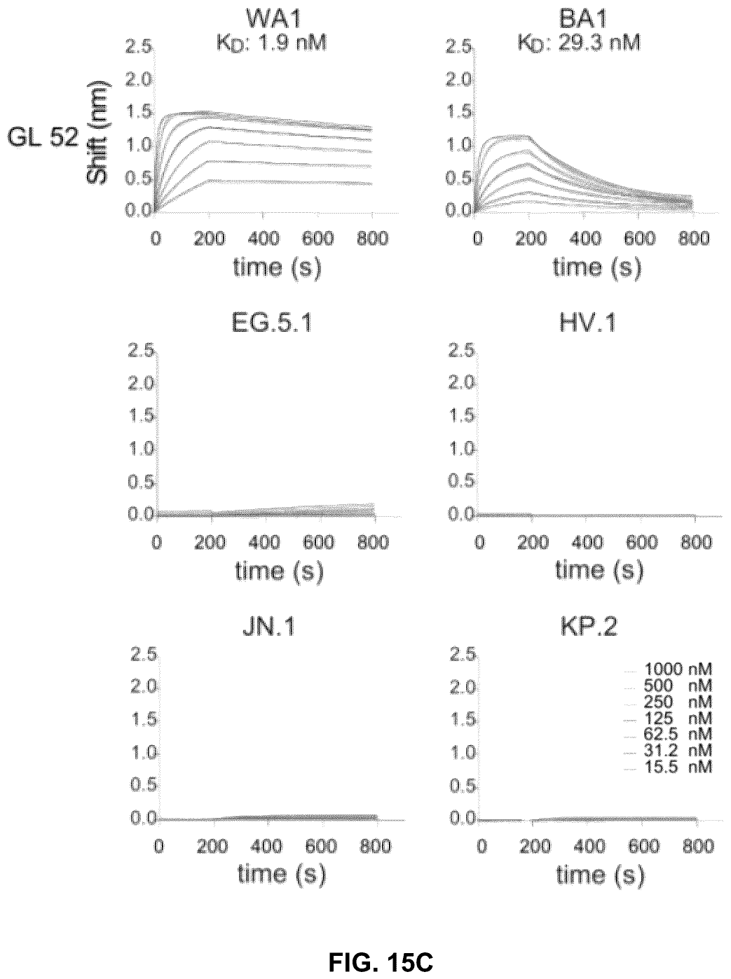

Previous work indicates somatic hypermutations F281 and Y57F in the IGHV3-66/IGHV3-53 public clonotypes led to affinity maturation and Omicron RBD binding. However, we did not observe side chains of these amino acids interacting with RBD, and reversion of these amino acids to germline (128F and F57Y) did not lead to a reduction or abrogation of binding to BA.1 Omicron RBD (FIG. 15A, FIG. 15B). We determined the binding affinity of the mature and germline Fab-52 to variant RBD proteins. The mature Fab-52 bound all the variant RBD proteins in nanomolar affinity from highest to lowest order as WA1/BA.1 (0.6 nM)>EG.5.1 (10 nM)>HV.1 (12.2 nM)>JN.1 (28 nM)>KP.2 (139 nM) (FIG. 15C). Germline Fab-52 bound WA1 (1.9 nM) and BA.1 (29.3 nM) RBD with nanomolar affinity, but lost binding to later Omicron variants EG.5.1, HV.1, JN.1 and, KP.2 (FIG. 15C). These results highlight the recruitment of some broadly cross-reactive, protective, and public B cell clones targeting class I/II RBD epitope to the germinal center following bivalent booster vaccination.

### DISCUSSION

We evaluated the GC B cell response to a bivalent (mRNA1273.214) SARS-CoV-2 vaccine booster in humans. All the participants responded to bivalent mRNA-1273.214 boosting based on frequencies of antibody-secreting S⁺ PBs in blood and increased serum binding titres to S protein (WA1/2020, BA.1). We observed a robust S⁺ GC response in the majority of vaccinees upon boosting with the bivalent vaccine. All the S⁺ GC B cell clones bound ancestral S-protein, highlighting potential recall responses recruited to the GC with no or very limited variant-specific naïve responses, possibly due to inclusion of the ancestral S protein (WA1/2020)-encoding mRNA to the bivalent booster. A major fraction (60.2%) of the RBD-targeting clones were extensively cross-reactive (WA1⁺BA.1⁺XBB.1.5⁺) with a single clone encoding mAb-52, which potently cross-neutralized newer Omicron variants through KP.2. mAb-52 targeted the class I/II RBD epitope, utilized a public clonotype IGHV3-66, and protected hamsters challenged with a distant EG.5.1 variant. These results highlight that bivalent booster vaccination can recruit some broadly neutralizing, public clones, indicating wider applicability and success at eliciting such responses in vaccinees. We note that the observed mAb-52 response was rare due to limited number of clones analyzed from a single time point FNA. Taken together, booster vaccination with variant-derived S-encoding mRNA could broaden the elicited responses by minimizing recruitment of naïve ancestral (WA1) S-binding clones. Furthermore, it is not surprising to observe a dominant antigenically imprinted serological responses as reported by several studies, owing to the addition of ancestral S-encoding mRNA to the vaccine. Nonetheless, this type of imprinting effect was seen previously with serum antibodies that gained cross-reactive neutralizing activity against distantly related Sarbecoviruses after boosting monovalently with BA.5 or XBB.1.5 mRNA vaccines. While a recent study indicated bivalent vaccination led to elicitation of heightened IgG4 subclass sera responses, we did not observe such IgG4 skewing of germinal center B cell responses at week 8 in all our participants. Another study recently reported that the JN.1 variant evades majority of antibodies utilizing IGHV3-53/66, whereas our study shows that mAb-52, also derived from IGHV3-66, broadly neutralizes evolved variants of concern, JN.1 and KP.2. These results suggest an affinity matured public clonotype is amenable to recruitment and further affinity maturation to neutralize Omicron-lineage descendants that evolve in the future. Eliciting such broad cross-neutralizing antibody responses like mAb-52 is the primary goal of a variant-derived vaccination that we could successfully capture in the GC B cell compartment of the lymph nodes following bivalent SARS-CoV-2 booster vaccination.

### Materials and Methods

### Sample Collection, Preparation, and Storage

Nine healthy volunteers were enrolled, of whom all provided axillary LN (Table 1). Blood samples were collected in ethylenediaminetetraacetic acid (EDTA) evacuated tubes (BD), and peripheral blood mononuclear cells (PBMC) were enriched by density gradient centrifugation over Lymphopure (BioLegend). The residual red blood cells were lysed with ammonium chloride lysis buffer, washed with PBS supplemented with 2% FBS and 2 mM EDTA (P2), and PBMC were immediately used or cryopreserved in 10% dimethylsulfoxide (DMSO) in FBS. Ultrasound-guided FNA of axillary LNs was performed by a radiologist. LN dimensions and cortical thickness were measured, and the presence and degree of cortical vascularity and location of the LN relative to the axillary vein were determined prior to each FNA. For each FNA sample, six passes were made under continuous real-time ultrasound guidance using 22- or 25-gauge needles, each of which was flushed with 3 mL of RPMI 1640 supplemented with 10% FBS and 100 U/mL penicillin/streptomycin, followed by three 1-mL rinses. Red blood cells were lysed with ammonium chloride buffer (Lonza), washed with P2, and immediately used or cryopreserved in 10% DMSO in FBS. Participants reported no adverse effects from phlebotomies or serial FNAs.

### Cell Lines

Expi293F cells were cultured in Expi293 Expression Medium (Gibco).

Vero cells expressing human ACE2 and TMPRSS2 (Vero-hACE2-hTMPRSS2) were cultured at 37° C. in Dulbecco's Modified Eagle medium (DMEM) supplemented with 10% fetal bovine serum (FBS), 10 mM HEPES (pH 7.3), 100 U/mL of penicillin, 100 μg/ml of streptomycin, and 10 μg/ml of puromycin. Vero cells expressing TMPRSS2 (Vero-hTMPRSS2) were cultured at 37° C. in Dulbecco's Modified Eagle medium (DMEM) supplemented with 10% fetal bovine serum (FBS), 10 mM HEPES (pH 7.3), 100 U/mL of penicillin, 100 μg/ml of streptomycin, and 5 μg/ml of blasticidin.

mAb-52
mAb-52 heavy chain sequence
(SEQ_ID_NO: 1)
ATGGGATGGTCATGTATCATCCTTTTTCTAGTAGCAACTGCAAC
CGGTGTCCAGTGTGAGGTGCAACTGGTGCAGTCTGGGGGAGACT
TGGCCCCGCCGGGGGGGTCCCTGAGACTCTCCTGTGAAGCCTCT
GGAATCATCGTCAGTAGCAACTACATGACCTGGGTCCGCCAGGC
TCCGGGGAAGGGGCTGGAGTGGGTCTCACTTATTTTTGCCGGTG
GAAGTACGTTCTACGCAGACTCCGTGAAGGGCCGATTCACCGTC
TCCAGAGACAATTCCAAGAATACGCTGTATCTTCAAATGAGCAG
CCTGAAGCCTGAGGACACGGCTGTCTATTTCTGTGCGAGAGATC
TTCGGGAAATGGGGGGACTTGACTTCTGGGGCCAGGGAGCCCTG
GTCACCGTCTCCTCAGCGTCGACCAAGGGCCCATCGGTCTTCCC
CCTGGCACCCTCCTCCAAGAGCACCTCTGGGGGCACAGCGGCCC
TGGGCTGCCTGGTCAAGGACTACTTCCCCGAACCTGTGACGGTC
TCGTGGAACTCAGGCGCCCTGACCAGCGGCGTGCACACCTTCCC
GGCTGTCCTACAGTCCTCAGGACTCTACTCCCTCAGCAGCGTGG
TGACCGTGCCCTCCAGCAGCTTGGGCACCCAGACCTACATCTGC
AACGTGAATCACAAGCCCAGCAACACCAAGGTGGACAAGAGAGT
TGAGCCCAAATCTTGTGACAAAACTCACACATGCCCACCGTGCC
CAGCACCTGAACTCCTGGGGGGACCGTCAGTCTTCCTCTTCCCC
CCAAAACCCAAGGACACCCTCATGATCTCCCGGACCCCTGAGGT
CACATGCGTGGTGGTGGACGTGAGCCACGAANACCCTGAGGTCA
AGTTCAACTGGNACGTNNNGGNNNNNNGNGCATAA.
mAb-52 light chain sequence
(SEQ_ID_NO: 2)
ATGGGATGGTCATGTATCATCCTTTTTCTAGTAGCAACTGCAAC
CGGTTCCAGATGCGACATCCAGATGACCCAGTCTCCATCTTCCG
TGTCTGCATCTATTGGAGACACAGTCACTATCACTTGTCGGGCG
AGTCAGGGTATTCCCAGCTGGGTAGCCTGGTATCAGCAGAAACC
TGGTAAAGCCCCTAAGCTCCTCATTTATGGTGCATCCAATTTGC
AGCGTGGGGTCCCATCAAGGTTCAGCGGCAGTGGATCTGGGACA
GATTTCACTCTCACCATCAGCAGCCTGCAGCCTGAAGATCTTGC
AGTTTACTATTGTCATCAGTCTGACAGTCTTCCCGGGATTTTCG
GCGGAGGGACAAAGGTGGAGATCAAACGTACGGTGGCTGCACCA
TCTGTCTTCATCTTCCCGCCATCTGATGAGCAGTTGAAATCTGG
AACTGCCTCTGTTGTGTGCCTGCTGAATAACTTCTATCCCAGAG
AGGCCAAAGTACAGTGGAAGGTGGATAACGCCCTCCAATCGGGT
AACTCCCAGGAGAGTGTCACAGAGCAGGACAGCAAGGACAGCAC
CTACAGCCTCAGCAGCACCCTGACGCTGAGCAAAGCAGACTACG
AGAAACACAAAGTCTACGCCTGCGAAGTCACCCATCAGGGCCTG
AGCTCGCCCGTCACAAAGAGCTTCAACAGGGGAGAGTGTTAG.
mAb-52 heavy chain amino acids
(SEQ_ID_NO: 3)
MTWVRQAPGKGLEWVSLIFAGGSTFYADSVKGRFTVSRDNSKNT
LYLQMSSLKPEDTAVYFCARDLREMGGLDFWGQGALVTVSS.
mAb-52 light chain amino acids-
(SEQ_ID_NO: 4)
MTQSPSSVSASIGDTVTITCRASQGIPSWVAWYQQKPGKAPKLL
IYGASNLQRGVPSRFSGSGSGTDFTLTISSLQPEDLAVYYCHQS
DSLPGIFGGGTKVEIK

### Antigens

Recombinant soluble spike protein(S) from WA1/2020 (2P), B.1.351 (2P), B.1.617.2 (2P), BA.1 (6P) strains of SARS-CoV-2 and their Avi-tagged counterparts were expressed as previously described. Briefly, mammalian cell codon-optimized nucleotide sequences coding for the soluble ectodomain of S (GenBank: MN908947.3, amino acids 1-1213) including a C-terminal thrombin cleavage site, T4 foldon trimerization domain, and hexahistidine tag (2P version)/octa histag (6P version) were cloned into mammalian expression vector pCAGGS. The S sequences were modified to remove the polybasic cleavage site (RRAR to A in WA1 and RRAR to GSAS in BA.1) and 2P (K986P and V987P), 6P (F817P, A892P, A899P, A942P, K986P and V987P). For expression of Avi-tagged variants, the CDS of PCAGGS vector containing the sequence for the relevant soluble S was modified to encode 3′ Avitag insert after the HIS tag (5′-HIS tag-GGCTCCGGGCTGAACGACATCTTCGAAGCCCAGAAGATTGAGTGGCATGAG-Stop-3′ (SEQ_ID_NO: 5); HIS tag-GSGLNDIFEAQKIEWHE-Stop (SEQ_ID_NO: 6)). Recombinant proteins were produced in Expi293F cells (ThermoFisher) by transfection with purified DNA using the ExpiFectamine 293 Transfection Kit (ThermoFisher). Supernatants from transfected cells were harvested 3 days post-transfection, and recombinant proteins were purified using Ni-NTA agarose (ThermoFisher), then buffer exchanged into phosphate buffered saline (PBS) and concentrated using Amicon Ultracel centrifugal filters (EMD Millipore). To biotinylate Avi-tagged S variants, the S-Avitag substrates were diluted to 40 UM and incubated for 1 h at 30° C. with 15 μg/ml BirA enzyme (Avidity) in 0.05 M bicine buffer at pH 8.3 supplemented with 10 mM ATP, 10 mM MgOAc, and 50 UM biotin. The protein was then concentrated/buffer exchanged with PBS using a 100 kDa Amicon Ultra centrifugal filter (MilliporeSigma).

To generate antigen probes for flow cytometry staining and sorting, trimeric BirA-biotinylated recombinant S from WA1/2020 or BA.1 (mRNA-1273) were incubated with a 1.04-fold molar excess of BV421-, BV650-, or PE-conjugated streptavidin (BioLegend) on ice, with three equal additions of S spaced every 15 min. Fifteen min after the third S addition, D-biotin was added in 6-fold molar excess to streptavidin to block any unoccupied biotin binding sites. SA-PE-Cy5 was blocked with a 6-fold molar excess of D-biotin and used as a background staining control. Bovine serum albumin (BSA) was biotinylated using the EZ-Link Micro NHS-PEG4-Biotinylation Kit (Thermo Fisher); excess unreacted biotin was removed using 7-kDa Zeba desalting columns (Pierce).

### ELISpot Assay

Wells of a microtiter plate were coated with recombinant S from the WA1/2020, B.1.351, B.1.617.2, BA.1, BSA or pooled anti-K and anti-A light chain antibodies (Cellular Technology Limited). Direct ex-vivo ELISpot assays were performed to determine the number of total, recombinant S-binding IgG- and IgA-secreting cells present in PBMC and enriched BMPC samples using IgG/IgA double-color ELISpot Kits (Cellular Technology Limited) according to the manufacturer's instructions. Plates were analyzed using an ELISpot counter (Cellular Technology Limited).

### ELISA

Assays were performed in 96-well MaxiSorp plates (Thermo Fisher) coated with 100 μL of recombinant SARS-CoV-2 S from WA1/2020 (2P), BA.1 (6P) and RBDs from WA1, BA.1, XBB.1.5 strains of SARS-CoV-2 bovine serum albumin diluted to 1 μg/ml in PBS, and plates were incubated at 4° C. overnight. Plates then were blocked with 10% FBS and 0.05% Tween 20 in PBS. Plasma or purified monoclonal antibodies were diluted serially starting at 1:30 or at fixed concentration of 10 μg/ml respectively in blocking buffer and added to the plates. Plates were incubated for 90 min at room temperature and then washed 3 times with 0.05% Tween 20 in PBS. Goat anti-human IgG-HRP secondary antibody (goat polyclonal, Jackson ImmunoResearch, 109-035-088, 1:2,500) was diluted in blocking buffer before adding to plates and incubating for 60 min at room temperature. Plates were washed 3 times with 0.05% Tween 20 in PBS and 3 times with PBS before the addition of o-phenylenediamine dihydrochloride peroxidase substrate (Sigma-Aldrich). Reactions were stopped by the addition of 1 M hydrochloric acid. Optical densities were measured at 490 nm.

### Flow Cytometry and Cell Sorting

Staining for flow cytometry analysis was performed using cryo-preserved FNA samples. For analysis, FNA samples were incubated for 30 min on ice with purified CD16 (3G8, BioLegend, 1:100), CD32 (FUN-2, BioLegend, 1:100), CD64 (10.1, BioLegend, 1:100) and PD-1-BB515 (EH12.1, BD Horizon, 1:100) in P2, washed twice, then stained for 30 min on ice with WA1/2020 probes pre-conjugated to SA-APC and SA-APC-Fire 750, BA.1 probes pre-conjugated to SA-BV421 and SA-BV650, biotin-saturated SA-PE-Cy5, IgG-BV480 (goat polyclonal, Jackson ImmunoResearch, 1:100), IgA-FITC (M24A, Millipore, 1:500), CD8-A532 (RPA-T8, Thermo, 1:100), CD38-BB700 (HIT2, BD Horizon, 1:500), CD20-Pacific Blue (2H7, 1:400), CD4-Spark Violet 538 (SK3, 1:400), IgM-BV605 (MHM-88, 1:100), CD19-BV750 (HIB19, 1:100), IgD-BV785 (IA6-2, 1:200), CXCR5-PE-Dazzle 594 (J252D4, 1:50), CD14-PerCP (HCD14, 1:50), CD71-PE-Cy7 (CY1G4, 1:400), CD27-PE-Fire 810 (O323, 1:200), CD3-APC-Fire 810 (SK7, 1:50), and Zombie NIR (all BioLegend) diluted in Brilliant Staining buffer (BD Horizon). Cells were washed twice with P2, fixed for 1 h at 25° C. using the True Nuclear fixation kit (BioLegend), washed twice with True Nuclear Permeabilization/Wash buffer, stained with Ki-67-BV711 (Ki-67, BioLegend, 1:200), Blimp1-PE (646702, R&D, 1:100), FoxP3-Spark 685 (206D, BioLegend, 1:200), and Bcl6-R718 (K112-91, BD Horizon, 1:200) for 1 h at 25° C., and washed twice with True Nuclear Permeabilization/Wash buffer. Samples were resuspended in P2 and acquired on an Aurora using SpectroFlo v2.2 (Cytek). Flow cytometry data were analyzed using FlowJo v10 (Treestar).

For sorting PB, PBMC collected 1 week post-boost were incubated for 30 min on ice with purified CD16 (3G8, BioLegend, 1:100), CD32 (FUN-2, BioLegend, 1:100), and CD64 (10.1, BioLegend, 1:100), then stained for 30 min on ice with CD4-Spark UV 387 (SK3, 1:200), CD20-Pacific Blue (2H7, 1:400), CD71-FITC (CY1G4, 1:200), IgD-PerCP-Cy5.5 (IA6-2, 1:200), CD19-PE (HIB19, 1:200), CXCR5-PE-Dazzle 594 (J252D4, 1:50), CD38-PE-Fire 810 (HIT2, 1:200), CD14-A700 (HCD14, 1:200), and Zombie NIR (all BioLegend) diluted in P2. Cells were washed twice, and PB (live singlet CD4⁻CD14⁻CD19⁺IgD^{lo} CD20^{lo}CD38⁺CXCR51^{lo}CD71⁺) were sorted using a Bigfoot (Invitrogen) into Buffer RLT Plus (Qiagen) supplemented with 143 mM β-mercaptoethanol (Sigma-Aldrich) and immediately frozen on dry ice.

For sorting memory B cells, PBMC collected at baseline and 17 weeks after boosting were incubated for 30 min on ice with purified CD16 (3G8, BioLegend, 1:100), CD32 (FUN-2, BioLegend, 1:100), and CD64 (10.1, BioLegend, 1:100), then stained for 30 min on ice with CD4-Spark UV 387 (SK3, 1:200), IgD-BV421 (IA6-2, 1:200), CD3-FITC (HIT3a, 1:200), CD19-PE (HIB19, 1:200), CD27-PE-Fire 810 (O323, 1:200), CD14-A700 (HCD14, 1:200), and Zombie NIR (all BioLegend) diluted in P2. Cells were washed twice, and MBC (live singlet CD3-CD4⁻CD14⁻CD19⁺IgD^{lo}) were sorted using a Bigfoot (Invitrogen) into Buffer RLT Plus (Qiagen) supplemented with 143 mM β-mercaptoethanol (Sigma-Aldrich) and immediately frozen on dry ice.

For bulk sorting GC B cells and LNPCs, lymph node FNA samples collected 8 weeks post-boosting were incubated for 30 min on ice with purified CD16 (3G8, BioLegend, 1:100), CD32 (FUN-2, BioLegend, 1:100), and CD64 (10.1, BioLegend, 1:100) in P2, then stained for 30 min on ice with PD-1-BB515 (EH12.1, BD Horizon, 1:100), CD20-Pacific Blue (2H7, 1:400), CD19-BV750 (HIB19, 1:100), IgD-PerCP-Cy5.5 (IA6-2, 1:200), CD71-PE (CY1G4, 1:400), CXCR5-PE-Dazzle 594 (J252D4, 1:50), CD38-PE-Cy7 (HIT2, 1:200), CD4-A700 (SK3, 1:400), and Zombie Aqua (all BioLegend) diluted in Brilliant Staining Buffer (BD Horizon). Cells were washed twice, and total GC B cells (live singlet CD4⁻CD19⁺IgD^{lo}CD20⁺CD38^{int}CXCR5⁺CD71⁺) and LNPCs (live singlet CD4⁻CD19⁺IgD^{lo}CD20^{lo}CD38⁺CXCR51^{lo}CD71⁺) were sorted using a Bigfoot (Invitrogen) into Buffer RLT Plus (Qiagen) supplemented with 143 mM β-mercaptoethanol (Sigma-Aldrich) and immediately frozen on dry ice.

### Single-Cell RNA-Seq Library Preparation and Sequencing

LN FNA samples were processed using the following 10× Genomics kits: Chromium Next GEM Single Cell 5′ Kit v2 (PN-1000263); Chromium Next GEM Chip K Single Cell Kit (PN-1000286); BCR Amplification Kit (PN-1000253); Dual Index Kit TT Set A (PN-1000215). Chromium Single Cell 5′ Gene Expression Dual Index libraries and Chromium Single Cell V(D)J Dual Index libraries were prepared according to manufacturer's instructions. Both gene expression and V(D)J libraries were sequenced on a Novaseq S4 (Illumina), targeting a median sequencing depth of 50,000 and 5,000 read pairs per cell, respectively.

### Bulk BCR Library Preparation and Sequencing

RNA was purified from sorted IgD^{lo}PBMCs (MBC), PB, GC B cells, LNPCs using the RNeasy Plus Micro kit (Qiagen). Reverse transcription, unique molecular identifier (UMI) barcoding, cDNA amplification (New England Biolabs #E6421), and Illumina linker addition to B cell heavy chain transcripts were performed using the human heavy chain only primers of NEBNext Immune Sequencing Kit (New England Biolabs #E6320) according to the manufacturer's instructions (provided upon request). High-throughput 2×300 bp paired-end sequencing was performed on the Illumina MiSeq platform with a 30% PhiX spike-in according to manufacturer's recommendations, except for performing 325 cycles for read 1 and 275 cycles for read 2.

### Preprocessing of Bulk Sequencing BCR Reads

Preprocessing of demultiplexed pair-end reads was performed using pRESTO v.0.6.2 as previously described, with the exception that sequencing errors were corrected using the UMIs as they were without additional clustering (Table 2).

TABLE 2
Processing of BCR reads from bulk sequencing.

| | | | | | Sequence Count | | | |
|---|---|---|---|---|---|---|---|---|
| | | | | | | | Post-QC | Unique |
| | | | | | | | Productive | Heavy |
| | | | | Cell | Input | Preprocessed | Heavy | Chain |
| Participant | Timepoint | Tissue | Sorting | Count | Reads | Reads | Chains | VDJs |
| 382-65 | d 0 | Blood | IgDlo | 250464 | 1474888 | 9670 | 8003 | 5268 |
| 382-65 | d 8 | Blood | PB | 25078 | 1629948 | 10858 | 8952 | 4265 |
| 382-65 | d 57 | LN | GC B | 28569 | 995015 | 5451 | 4295 | 2563 |
| 382-65 | d 57 | LN | LNPC | 4146 | 1309457 | 15396 | 12329 | 4266 |
| 382-65 | d 121 | Blood | IgDlo | 494579 | 1972488 | 17182 | 14866 | 9459 |
| 382-67 | d 0 | Blood | IgDlo | 102255 | 1857265 | 12973 | 11231 | 7022 |
| 382-67 | d 8 | Blood | PB | 21585 | 1733950 | 6762 | 5446 | 3506 |
| 382-67 | d 57 | LN | GC B | 23203 | 949129 | 1995 | 1512 | 891 |
| 382-67 | d 57 | LN | LNPC | 3266 | 1295174 | 6675 | 5160 | 2217 |
| 382-67 | d 121 | Blood | IgDlo | 219064 | 1752127 | 11871 | 10300 | 6799 |
| 382-69 | d 0 | Blood | IgDlo | 104705 | 1586882 | 12487 | 10657 | 6899 |
| 382-69 | d 57 | LN | GC B | 1604 | 1052477 | 283 | 154 | 92 |
| 382-69 | d 57 | LN | LNPC | 302 | 1053350 | 966 | 649 | 299 |
| 382-69 | d 121 | Blood | IgDlo | 85444 | 1985842 | 16447 | 14578 | 9126 |
| 382-70 | d 0 | Blood | IgDlo | 230258 | 2018408 | 19335 | 17377 | 9963 |
| 382-70 | d 8 | Blood | PB | 59615 | 1945779 | 27737 | 25634 | 14410 |
| 382-70 | d 57 | LN | GC B | 4801 | 1001226 | 996 | 649 | 341 |
| 382-70 | d 57 | LN | LNPC | 1644 | 954374 | 4563 | 3567 | 1481 |
| 382-70 | d 121 | Blood | IgDlo | 272610 | 2156732 | 64838 | 58829 | 23979 |
| 382-71 | d 0 | Blood | IgDlo | 295954 | 1944729 | 17354 | 15430 | 10148 |
| 382-71 | d 8 | Blood | PB | 51971 | 1852169 | 43305 | 39101 | 12593 |
| 382-71 | d 121 | Blood | IgDlo | 300504 | 1736137 | 18442 | 16869 | 11592 |

### Preprocessing of 10× Genomics Single-Cell BCR Reads

Demultiplexed pair-end FASTQ reads were preprocessed using Cell Ranger v.6.0.1 as previously described (Table 3).

TABLE 3
Processing of BCR and 5′ gene expression data from scRNA-seq

| | | | | | | 5′ gene expression | | | |
|---|---|---|---|---|---|---|---|---|---|
| | | | | BCR | | | | Median | Median |
| | | | | Pre-QC | Post-QC | Pre-QC | Post-QC | number | number |
| | | | | number | number | number | number | of UMIs | of genes |
| Participant | Timepoint | Tissue | Replicate | of cells | of cells | of cells | of cells | per cell | per cell |
| 382-65 | d 57 | LN | 1 | 2990 | 2750 | 9648 | 9082 | 3646 | 1419.5 |
| | d 57 | LN | 2 | 2548 | 2350 | 8465 | 7907 | 3740 | 1410 |
| 382-67 | d 57 | LN | 1 | 3266 | 2938 | 7360 | 6995 | 3536 | 1447 |
| | d 57 | LN | 2 | 3710 | 3293 | 7409 | 7086 | 3382.5 | 1395 |
| 382-69 | d 57 | LN | 1 | 4638 | 4205 | 9358 | 8680 | 3474 | 1404 |
| | d 57 | LN | 2 | 8341 | 6494 | 22007 | 21591 | 776 | 514 |
| 382-70 | d 57 | LN | 1 | 1508 | 1400 | 8982 | 8808 | 3228 | 1244 |
| | d 57 | LN | 2 | 1399 | 1330 | 8733 | 8513 | 3392 | 1312 |
| 382-71 | d 57 | LN | 1 | 2113 | 2018 | 8692 | 8563 | 3732 | 1444 |
| | d 57 | LN | 2 | 2091 | 2002 | 8830 | 8690 | 3806.5 | 1456 |

### V(D)J Gene Annotation and Genotyping

Initial germline V(D)J gene annotation was performed on the preprocessed BCRs using IgBLAST v.1.17.1 with the deduplicated version of IMGT/V-QUEST reference directory release 202113-2. Isotype annotation for 10× Genomics sequences was pulled from the ‘c_call’ column in the ‘filtered_contig_annotations.csv’ files outputted by Cell Ranger. Further sequence-level and cell-level quality controls were performed as previously described. Individualized genotypes were inferred based on sequences that passed all quality controls using TIgGER v.1.0.0 and used to finalize V(D)J annotations. Sequences annotated as non-productively rearranged by IgBLAST were removed from further analysis.

### Clonal Lineage Inference

B cell clonal lineages were inferred on a by-individual basis based on productively rearranged sequences as previously described. Briefly, heavy chain-based clonal inference was performed by partitioning the heavy chains of bulk and single-cell BCRs based on common V and J gene annotations and CDR3 lengths, and clustering the sequences within each partition hierarchically with single linkage based on their CDR3s. Sequences within 0.15 normalized Hamming distance from each other were clustered as clones. Following clonal inference, full-length clonal consensus germline sequences were reconstructed using Change-O v.1.0.2. Within each clone, duplicate IMGT-aligned V(D)J sequences from bulk sequencing were collapsed using Alakazam v1.1.0 except for duplicates derived from different time points, tissues, B cell compartments, isotypes, or biological replicates.

### BCR Analysis

For B cell compartment labels, gene expression-based cluster annotation was used for single-cell BCRs; and FACS-based sorting and magnetic enrichment were used for bulk BCRs, except that IgD^{lo} enriched B cells from PMBC were labelled MBCs. For analysis involving the memory compartment, the memory sequences were restricted to those from blood. A heavy chain-based B cell clone was considered S-specific if it contained any sequence corresponding to a recombinant monoclonal antibody that was synthesized based on the single-cell BCRs and that tested positive for S-binding. Somatic hypermutation (SHM) frequency was calculated for each heavy chain sequence using SHazaM v.1.0.2 by counting the number of nucleotide mismatches from the germline sequence in the variable segment leading up to the CDR3.

### Processing of 10× Genomics Single-Cell 5′ Gene Expression Data

Demultiplexed pair-end FASTQ reads were first preprocessed on a by-sample basis and samples were subsequently subsampled to the same effective sequencing length and aggregated using Cell Ranger v.6.0.1 as previously described. Quality control was performed on the aggregate gene expression matrix consisting of 99,484 cells and 36,601 features using SCANPY v.1.7.2. Briefly, to remove presumably lysed cells, cells with mitochondrial content greater than 20% of all transcripts were removed. To remove likely doublets, cells with more than 8,000 features or 80,000 total UMIs were removed. To remove cells with no detectable expression of common endogenous genes, cells with no transcript for any of a list of 34 housekeeping genes were removed. The feature matrix was subset, based on their biotypes, to protein-coding, immunoglobulin, and T cell receptor genes that were expressed in at least 0.05% of the cells in any sample. The resultant feature matrix contained 14,796 genes. Finally, cells with detectable expression of fewer than 200 genes were removed. After quality control, there were a total of 95,915 cells from 10 single-cell samples (Table 3).

### Single-Cell Gene Expression Analysis

Transcriptomic data was analyzed using SCANPY v.1.7.2 as previously described with minor adjustments suitable for the current data. Briefly, overall clusters were first identified using Leiden graph-clustering with resolution 0.18 (FIG. 7A, Table 4). UMAPs were faceted by participant and inspected for convergence to assess whether there was a need for integration. Cluster identities were assigned by examining the expression of a set of marker genes for different cell types (FIG. 7B). To remove potential contamination by platelets, 143 cells with a log-normalized expression value of >2.5 for PPBP were removed. Cells from the overall B cell cluster were further clustered to identify B cell subsets using Leiden graph-clustering resolution 0.25 (FIG. 2A, Table 4). Cluster identities were assigned by examining the expression of a set of marker genes for different B cell subsets (FIG. 7C, FIG. 7D) along with the availability of BCRs. A group of 813 cells displaying expression signatures of both naïve and memory B cells was further clustered into 370 naïve B cells and 443 MBCs. Despite being clustered with B cells during overall clustering, one group tended to have both BCRs and relatively high expression levels of CD2 and CD3E. Within this group, 4 cells with CD3E expression below group mean and RGS13 expression above group mean were assigned to be GC B cells, whereas the rest “B & T”. Two unassigned groups tended to have no BCRs and no distinct expression signature of known B cell subsets. The “B & T” and unassigned groups were excluded from the final B cell clustering. Heavy chain SHM frequency and isotype usage of the B cell subsets were inspected for consistency with expected values to further confirm their assigned identities.

Selection of Single-Cell BCRs from GC B Cell or LNPC Clusters for Expression

Single-cell gene expression analysis was performed on a by-participant basis. Clonal inference was performed based on paired heavy and light chains. From every clone containing a cell from the GC B cell cluster and/or the LNPC cluster, one GC B cell or LNPC was selected. For selection, where a clone spanned both the GC B cell and LNPC compartments, a compartment was first randomly selected. Within that clone, the cell with the highest heavy chain UMI count was then selected, breaking ties based on IGHV SHM frequency. In all selected cells, native pairing was preserved. The selected BCRs were curated as previously described prior to synthesis.

Transfection for Recombinant mAbs and Fab Production

Selected pairs of heavy and light chain sequences were synthesized by GenScript and sequentially cloned into IgG1, Igκ/λ and Fab expression vectors. Heavy and light chain plasmids were co-transfected into Expi293F cells (Thermo Fisher Scientific) for recombinant mAb production, followed by purification with protein A agarose resin (GoldBio). Expi293F cells were cultured in Expi293 Expression Medium (Gibco) according to the manufacturer's protocol.

### Chimeric VSV-SARS-CoV-2 Neutralization Assay

The VSV-based neutralization assay was performed as described previously. The gene encoding spike of SARS-CoV-2 isolate WA1/2020 (with D614G mutation) was synthesized and replaced the native envelope glycoprotein of an infectious molecular clone of VSV, and resulting chimeric viruses expressing S protein from SARS-CoV-2 D614G was used for GFP reduction neutralization tests as previously described. Briefly, 2×10³ PFU of VSV-SARS-CoV-2-S_{Δ21} was incubated for 1 h at 37° C. with recombinant mAbs diluted to 10 μg/ml. Antibody-virus complexes were added to Vero E6 cells in 96-well plates and incubated at 37° C. for 7.5 h. Cells were subsequently fixed in 2% formaldehyde (Electron Microscopy Sciences) containing 10 mg/mL Hoechst 33342 nuclear stain (Invitrogen) for 45 min at room temperature, when fixative was replaced with PBS. Images were acquired with an Cytation C10 automated microscope (BioTek) using the DAPI and GFP channels to visualize nuclei and infected cells (i.e., eGFP-positive cells), respectively (4× objective, 4 fields per well, covering the entire well). Images were analyzed using the Gen5 3.12 software's Data Reduction tool (BioTek). GFP-positive cells were identified in the GFP channel following image preprocessing, deconvolution and subsequently counted within the Gen5 3.12 software. The sensitivity and accuracy of GFP-positive cell number determinations were validated using technical replicates. The percent infection reduction was calculated from wells to which no antibody was added. A background number of GFP-positive cells was subtracted from each well using an average value determined from at least 4 uninfected wells.

### Viruses

The WA1/2020 recombinant strain with D614G substitution was described previously. The BA.1 isolate (hCoV-19/USA/WI-WSLH-221686/2021) was obtained from an individual in Wisconsin as a mid-turbinate nasal swab. BA.2.86 (hCoV-1/USA/MI-UM-10052670540/2023), XBB.1.5 (hCoV-19/USA/MD-HP40900-PIDYSWHNUB/2022), JN.1 (hCoV-19/USA/CA-Stanford-165_S10/2023), HV.1 (hCoV-19/USA/CA-Stanford-165_S45/2023), and KP.2 (hCoV-19/USA/CA-Stanford-181_S33/2024) were generous gifts from A. Pekosz (Johns Hopkins), and M. Suthar (Emory University).

The EG.5.1 variant of SARS-CoV-2 (hCoV-19/USA/CA-Stanford-147_S01/2023) was propagated on Vero-hTMPRSS2 cells. The virus stocks were subjected to next-generation sequencing, and the S protein sequences were identical to the original isolates. The infectious virus titer was determined by plaque and focus-forming assay on Vero-hACE2-hTMPRSS2 or Vero-hTMPRSS2 cells.

### Hamster Challenge Studies

Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Five-week-old male hamsters (n=6/group) were obtained from Charles River Laboratories and housed in a biosafety level 3 facility at Washington University. One day prior to challenge with 104 plaque forming units (PFU) of EG.5.1, the animals received intraperitoneal 10 mg/kg of mAb-52 or isotype control (1G05) in PBS. Animal weights were measured daily for the duration of the experiment. Three days after the challenge, hamsters were euthanized and necropsied, and the left lung lobe, nasal wash and nasal turbinates were collected for virological analysis. These tissues were homogenized in 1.0 mL of DMEM, clarified by centrifugation (1,000×g for 5 min) and used for viral titer analysis by quantitative RT-PCR (RT-qPCR) using primers and probes targeting the N gene, and by plaque assay.

### Virus Titer Assays

Plaque assays were performed on Vero-hACE2-hTMPRSS2 cells in 24-well plates. Lung tissue and nasal turbinate homogenates were serially diluted 10-fold, starting at 1:10, in cell infection medium (DMEM supplemented with 2% FBS, 10 mM HEPES, and 2 mM L-glutamine). Two hundred and fifty microliters of the diluted homogenate were added to a single well per dilution per sample. After 1 h at 37° C., the inoculum was aspirated, the cells were washed with PBS, and a 1% methylcellulose overlay in MEM supplemented with 2% FBS was added. Seventy-two hours after virus inoculation, the cells were fixed with 4% formalin, and the monolayer was stained with crystal violet (0.5% w/v in 25% methanol in water) for 1 h at 20° C. The number of plaques were counted and used to calculate the PFU/mL. To quantify viral RNA levels in the homogenates, RNA was extracted from 100 μL homogenate, using an automated RNA extraction machine (KingFisher Flex) and the MagMax Viral Pathogen kit according to the manufacturer's recommendations. The RNA was eluted in 50 μL of water. Four microliters RNA was used RT-qPCR to detect and quantify N gene of SARS-CoV-2 using the TaqMan RNA-to-CT 1-Step Kit (Thermo Fisher Scientific) with the following primers and probes for the N-gene, Forward primer: ATGCTGCAATCGTGCTACAA (SEQ_ID_NO: 7); Reverse primer: GACTGCCGCCTCTGCTC (SEQ_ID_NO: 8); Probe:/56-FAM/TCAAG GAAC/ZEN/AACATTGCCAA (SEQ_ID_NO: 9)/3IABKFQ/. Viral RNA was expressed as gene copy numbers per mg for lung tissue homogenates and per mL for nasal turbinates, based on a standard included in the assay.

### Focus Reduction Neutralization Test (FRNT)

Serial dilutions of each mAb were incubated with 102 focus-forming units (FFU) of different SARS-CoV-2 strains (WA1/2020 D614G, BA.1, XBB.1.5, EG.5.1, BA.2.86, HV.1, JN.1, or KP.2) for 1 h at 37° C. Antibody-virus complexes were added to Vero-TMPRSS2 cell monolayers in 96-well plates and incubated at 37° C. for 1 h. Subsequently, cells were overlaid with 1% (w/v) methylcellulose in MEM. Plates were harvested 30 h (WA1/2020 D614G) or 65-70 h (Omicron strains) later by removing overlays and fixed with 4% PFA in PBS for 20 min at room temperature. Plates were washed and incubated with an oligoclonal pool of anti-S antibodies (SARS2-2, SARS2-11, SARS2-16, SARS2-31, SARS2-38, SARS2-57, and SARS2-71), and an additional oligoclonal pool of anti-S antibodies with Supplementary reactivity (SARS2-08, -09, -10, -13, -14, -17, -20, -26, 27, -28, -31, -41, -42, -44, -49, -62, -64, -65, and -67) were included for staining plates infected with Omicron strains. Plates were subsequently incubated with HRP-conjugated goat anti-mouse IgG (Sigma Cat #A8924, RRID: AB_258426) in PBS supplemented with 0.1% saponin and 0.1% bovine serum albumin. SARS-CoV-2-infected cell foci were visualized using TrueBlue peroxidase substrate (KPL) and quantitated on an ImmunoSpot microanalyzer (Cellular Technologies).

### Antibody Escape Mapping Using Deep Mutational Scanning

XBB.1.5 RBD deep mutational scanning libraries were designed as described previously. For antibody selection experiments, approximately 1 M transcription units of the library were incubated with 5.3 and 21.3 μg/ml of mAb-52 for 45 min at 37° C. These concentrations were determined using XBB.1.5 pseudovirus neutralization assay and were approximately IC99*4 and IC99*16 as measured on HEK-293T-ACE2 cells. After incubation virus-antibody mix was used to infect HEK-293T-ACE2 cells. Viral genomes were recovered for deep sequencing 12 hours after infection. Antibody escape was mapped using two independent XBB.1.5 RBD libraries. Mutation-level escape was determined by using non-neutralizable control as described previously and a biophysical model implemented in polyclonal package.

### Fab Generation

Fabs of mAb-52 used for binding studies were produced in-house as described previously. Heavy chain encoding plasmids were restriction digested with AgeI, SaII and VH region was gel extracted and subcloned by ligation into Fab expression vector. Genes encoding germline revertants of FAb-52 were synthesized by Genscript and subcloned into heavy chain encoding plasmids as described above. The sequence confirmed Fab heavy chain and light chain plasmids were co-transfected into Expi293F cells (Gibco) for expression and purified with HisPur Ni-NTA resin (Thermo Scientific).

### Biolayer Interferometry

Kinetic binding studies were performed on an Octet-R8 (Sartorius) instrument. Avi-tagged biotinylated RBD of SARS-CoV-2 variants WA1/2020, BA.1, EG.5.1, HV.1, JN.1, KP.2, KP.3 were produced inhouse. Octet™ SA-Biosensor tips (Sartorius) were pre-equilibrated in HBS supplemented with 0.05% Tween-20 and 1% BSA (kinetic buffer A) followed by loading of Avi-RBD proteins to 1.0 nm. Kinetic binding studies were performed in kinetic buffer A by monitoring Fabs association (200 s) and dissociation (600 s). Octet™ SA-Biosensors that were not loaded were used as reference sensor. Kinetic parameters of reference subtracted kinetic traces were calculated with Octet BLI analysis software v12.1 using a global fit 1:1 binding model. Traces were plotted with GraphPad Prism v10.

BLI competition studies were performed on an Octet Red instrument (ForteBio). Biotinylated Avi tagged WA1 RBD was loaded onto Streptavidin sensor tips (Sartorius) to 2 nm that were pre-equilibrated in kinetic buffer A. Following loading, mAb-52/1G05 (isotype) (250 nM) were monitored for binding for 200 s and followed by 200 s of competitive binding against 2B04 (class I/A) and ACE2 receptor (250 nM). Octet™ SA-Biosensors that were loaded and dipped in blank buffer were used as reference sensors. The relative shift in competitive mAb/receptor binding was quantified between 200-400 s relative to 1G05 isotype control following reference subtraction of kinetic traces. Traces were plotted with GraphPad Prism v10.

### Cryo Electron Microscopy Sample Preparation, Data Collection

The SARS-CoV-2 XBB.1.5 HexaPro spike (GISAID Accession ID-EPI_ISL_18416647) was mixed with Fab-52 at a concentration of 2 mg/mL, using a 1.5 molar excess of Fab, and incubated for 20 minutes at room temperature. Immediately before grid preparation, fluorinated octyl-maltoside was added to the complex at a final concentration of 0.02% wt/vol. Next, 3 μl aliquots were applied to UltrAuFoil gold R1.2/1.3 grids, which were blotted for 6 seconds at a blot force of 0, at 22° C. and 95% humidity. The samples were then plunge-frozen in liquid ethane using a Vitrobot Mark IV system (ThermoFisher Scientific). Imaging was conducted on a Titan Krios microscope operated at 300 kV and equipped with a 15 eV energy filter and a Gatan K3 direct electron detector. A total of 6,614 movie frames were captured, with a cumulative dose of 48.95 e−/Å²/s. Images were recorded at a magnification of 105,000, corresponding to a calibrated pixel size of 0.4125 Å/pixel, with a defocus range from −0.9 to −2.3 μm.

### Cryo Electron Microscopy Data Processing, Structure Modelling and Refinement

The movies were aligned and dose-weighted using the patch motion correction feature in cryoSPARC v4.3.1. The contrast transfer function (CTF) was estimated using Patch CTF, and particles were picked with cryoSPARC's template picker. The picked particles were extracted with a box size of 1024 pixels, with 4× binning, and subjected to a 2D classification. An initial model was generated from 625,012 selected particles, and the best class containing 267,722 was chosen for further analysis. After two rounds of non-uniform refinement, without imposed symmetry, the particles were subjected to 3D classification with six classes, where one class was selected for additional processing, containing 317,638 particles. These particles were re-extracted with a box size of 1024 pixels and 2× binning, followed by further rounds of non-uniform refinement that included local and global CTF refinement, resulting in a final global map with a nominal resolution of 2.58 Å. There was only one Fab with one of the protomers that was subjected to local refinement with a soft mask extended by 6 pixels and padded by 12 pixels encompassing the receptor binding domain (RBD) and Fab. This local refinement yielded a resolution of 3.11 Å. The two half-maps from this refinement were sharpened using DeepEMhancer. The reported resolutions are based on the gold-standard Fourier shell correlation criterion of 0.143.

The focused maps sharpened with DeepEMhancer were used for model building. The initial model was created using ModelAngelo and then manually refined with COOT. N-linked glycans were added manually in COOT using the glyco extension. The model underwent further refinement in Phenix, employing real-space refinement, and was validated using MolProbity (Table 5). The structural biology software was compiled and made available through SBGrid.

TABLE 4
scRNA-seq derived transcriptional cluster cell counts and frequencies

| | | | | | | | SARS-CoV-2 | |
|---|---|---|---|---|---|---|---|---|
| | Overall | Cell Count | | B cell | Cell Count | | S-binging cell count | |
| Participant | Cluster | (% of total cells) | | cluster | (% of B cells) | | (% in each B cell cluster) | |
| 382-65 | B | 6384 | (37.6%) | GC B | 496 | (10.9%) | 300 | (60.5%) |
| | CD4+ T | 9080 | (53.5%) | LNPC | 127 | (2.8%) | 115 | (90.6%) |
| | CD8+ T | 1048 | (6.2%) | MBC | 2965 | (65.4%) | 149 | (5.0%) |
| | NK | 125 | (0.7%) | Naïve | 943 | (20.8%) | 0 | (0.2%) |
| | Monocyte | 251 | (1.5%) | | | | | |
| | pDC | 98 | (0.6%) | | | | | |
| 382-67 | B | 6295 | (44.7%) | GC B | 880 | (14.8%) | 599 | (68.1%) |
| | CD4+ T | 5754 | (40.9%) | LNPC | 173 | (2.9%) | 143 | (82.7%) |
| | CD8+ T | 1567 | (11.1%) | MBC | 3889 | (65.3%) | 3 | (0.1%) |
| | NK | 198 | (1.4%) | Naïve | 1011 | (17%) | 0 | (0%) |
| | Monocyte | 205 | (1.5%) | | | | | |
| | pDC | 62 | (0.4%) | | | | | |
| 382-69 | B | 21340 | (70.8%) | GC B | 372 | (5.8%) | 200 | (54.6%) |
| | CD4+ T | 6655 | (22.1%) | LNPC | 474 | (7.4%) | 415 | (87.6%) |
| | CD8+ T | 1562 | (5.2%) | MBC | 1970 | (30.8%) | 5 | (0.3%) |
| | NK | 145 | (0.5%) | Naïve | 3585 | (56%) | 0 | (0%) |
| | Monocyte | 337 | (1.1%) | | | | | |
| | pDC | 92 | (0.3%) | | | | | |
| 382-70 | B | 2831 | (16.3%) | GC B | 132 | (5.3%) | 61 | (46.2%) |
| | CD4+ T | 12329 | (71.2%) | LNPC | 28 | (1.1%) | 21 | (75.0%) |
| | CD8+ T | 1827 | (10.5%) | MBC | 1620 | (64.7%) | 2 | (0.1%) |
| | NK | 123 | (0.7%) | Naïve | 723 | (28.9%) | 0 | (0%) |
| | Monocyte | 141 | (0.8%) | | | | | |
| | pDC | 70 | (0.4%) | | | | | |
| 382-71 | B | 4206 | (24.4%) | GC B | 138 | (3.7%) | 38 | (27.5%) |
| | CD4+ T | 11252 | (65.2%) | LNPC | 42 | (1.1%) | 33 | (78.6%) |
| | CD8+ T | 1512 | (8.8%) | MBC | 1768 | (47.3%) | 0 | (0%) |
| | NK | 102 | (0.6%) | Naïve | 1792 | (47.9%) | 0 | (0%) |

| | Monocyte | 103 | (0.6%) | | | | |
|---|---|---|---|---|---|---|---|
| | pDC | 78 | (0.5%) | | | | |
| Combined | B | 41056 | (41.9%) | GC B | 2018 | 1198 | (59.4%) |
| | CD4+ T | 45070 | (47.1%) | LNPC | 844 | 727 | (86.1%) |
| | CD8+ T | 7516 | (7.8%) | MBC | 12212 | 159 | (1.3%) |
| | NK | 693 | (0.7%) | Naïve | 8054 | 2 | (0.02%) |

| | | |
|---|---|---|
| Monocyte | 1037 | (1.1%) |
| pDC | 400 | (0.4%) |

TABLE 5
Cryo-EM data coIlection and refinement statistics
PDB ID 9E21 and EMD- 47426
Data Collection

| Grid type | UltrAuFoil gold R1.2/1.3 | |
|---|---|---|
| Microscope/voltage/detector | Titan Krios/300 kV/Gatan K3 | |
| Magnification | 105,000 | |
| Recording mode | counting | |
| Total dose | 48.95 | e−/Å²/s |
| Pixel size | 0.825 | Å/pixel |
| Defocus range | −0.9 to −2.3 | μm |

| | |
|---|---|
| No. micrographs used | 6,614 |
| Total particles picked | 630,579 |

Model Validation

| | |
|---|---|
| Composition (#) | |
| Chains | 4 |
| Atoms | 3285 |
| Residues | Protein: 424 Nucleotide: 0 |
| Ligands | NAG: 1 |

Bonds (RMSD)

| | |
|---|---|
| Length (Å) (# > 4sigma) | 0.004 (0) |
| Angles (°) (# > 4sigma) | 0.749 (0) |
| MolProbity score | 1.39 |
| Clash score | 3.40 |

Ramachandran plot (%)

| | |
|---|---|
| Outliers | 0.00 |
| Allowed | 3.83 |
| Favored | 96.17 |
| Rotamer outliers (%) | 0.00 |
| Cbeta outliers (%) | NA |

Peptide plane (%)

| | |
|---|---|
| Cis proline/general | 0.0/0.0 |
| Twisted proline/general | 0.0/0.0 |
| CaBLAM outliers (%) | 2.18 |

Data

| | |
|---|---|
| Lengths (Å) | 59.40, 74.25, 108.07 |
| Angles (°) | 90.00, 90.00, 90.00 |
| Supplied Resolution (Å) | 3.1 |
| Resolution Estimates (Å) | Masked |
| d FSC (half maps; 0.143) | 3.1 |
| d 99 (full/half1/half2) | 2.2/1.7/1.7 |
| d model | 2.0 |
| d FSC model (0/0.143/0.5) | 1.4/1.8/3.3 |
| Map min/max/mean | −0.00/2.31/0.03 |

Model vs. Data

| | |
|---|---|
| CC (mask) | 0.78 |
| CC (box) | 0.63 |
| CC (peaks) | 0.49 |
| CC (volume) | 0.78 |
| Mean CC for ligands | 0.83 |

1. A composition to treat a SARS-CoV-2 infection, the composition comprising an mAb-52 monoclonal antibody.

2. The composition of claim 1, wherein the mAb-52 monoclonal antibody comprises:
an IGHV3-66 heavy chain comprising the amino acid sequence
MTWVRQAPGKGLEWVSLIFAGGSTFYADSVKGRFTVSRDNSKNTLYL QMSSLKPEDTAVYFCARDLREMGGLDFWGQGALVTVSS (SEQ_ID_NO: 3) and variants thereof; and
a light chain comprising the amino acid sequence
MTQSPSSVSASIGDTVTITCRASQGIPSWVAWYQQKPGKAPKLLIYGA SNLQRGVPSRFSGSGSGTDFTLTISSLQPEDLAVYYCHQSDSLPGIFG GGTKVEIK (SEQ_ID_NO: 4) and variants thereof.

3. The composition of claim 1, wherein the mAb-52 monoclonal antibody targets a receptor binding domain (RBD) of the SARS-CoV-2 spike protein and competes with ACE2.

4. The composition of claim 3, wherein the mAb-52 monoclonal antibody targets a class I/II RBD epitope of the SARS-CoV-2 spike protein.

5. The composition of claim 4, wherein the mAb-52 monoclonal antibody binds the RBD of the SARS-CoV-2 spike protein with interactions comprising polar and hydrophobic interactions of CDRH1, CDRH2, CDRH3, CDRL1, and CDRL3.

6. The composition of claim 1, wherein the mAb-52 monoclonal antibody neutralizes a SARS-CoV-2 strain comprising the variants WA1, BA.1, XBB.1.5, EG.5.1, BA.2.86, HV.1, JN.1, and KP.2.

7. The composition of claim 1, further comprising a SARS-CoV-2 mRNA vaccine and a pharmaceutically acceptable carrier thereof.

8. The composition of claim 7, wherein the SARS-CoV-2 mRNA vaccine is selected from mRNA-1273.213, mRNA-1273.214, mRNA-1273.529, BNT162b2, and any combination thereof.

9. A method of treating a SARS-CoV-2 infection in a subject, the method comprising administering to the subject a therapeutically effective amount of a composition comprising an mAb-52 monoclonal antibody.

10. The method of claim 9, wherein the mAb-52 monoclonal antibody comprises:
an IGHV3-66 heavy chain comprising the amino acid sequence
MTWVRQAPGKGLEWVSLIFAGGSTFYADSVKGRFTVSRDNSKNTLYL QMSSLKPEDTAVYFCARDLREMGGLDFWGQGALVTVSS (SEQ_ID_NO: 3) and variants thereof; and
a light chain comprising the amino acid sequence
MTQSPSSVSASIGDTVTITCRASQGIPSWVAWYQQKPGKAPKLLIYGA SNLQRGVPSRFSGSGSGTDFTLTISSLQPEDLAVYYCHQSDSLPGIFG GGTKVEIK (SEQ_ID_NO: 4) and variants thereof.

11. The method of claim 9, wherein the mAb-52 monoclonal antibody is administered to the subject at a dose ranging from 5 mg/kg to 15 mg/kg.

12. The method of claim 11, wherein the mAb-52 monoclonal antibody is administered to a subject in need at a dose of 10 mg/kg.

13. The method of claim 9, wherein the mAb-52 monoclonal antibody targets a receptor binding domain (RBD) of the SARS-CoV-2 spike protein and competes with ACE2.

14. The method of claim 9, wherein the mAb-52 monoclonal antibody targets a class I/II RBD epitope of the SARS-CoV-2 spike protein.

15. The method of claim 9, wherein the mAb-52 monoclonal antibody neutralizes a SARS-CoV-2 strain comprising the variants WA1, BA.1, XBB.1.5, EG.5.1, BA.2.86, HV.1, JN.1, and KP.2.

16. The method of claim 9, wherein the composition further comprises a SARS-CoV-2 mRNA vaccine and a pharmaceutically acceptable carrier thereof.

17. The method of claim 16, wherein the SARS-CoV-2 mRNA vaccine is selected from mRNA-1273.213, mRNA-1273.214, mRNA-1273.529, BNT162b2, and any combination thereof.


SEQUENCE LISTING (17 sequences)

SEQ ID NO: 1 - DNA, 915 bp, synthetic construct
  source 1..915: mol_type = other DNA
atgggatggtcatgtatcatcctttttctagtagcaactgcaaccggtgtccagtgtgaggtgcaactggtgcagtctgggggagacttggccccgccgggggggtccctgagactctcctgtgaagcctctggaatcatcgtcagtagcaactacatgacctgggtccgccaggctccggggaaggggctggagtgggtctcacttatttttgccggtggaagtacgttctacgcagactccgtgaagggccgattcaccgtctccagagacaattccaagaatacgctgtatcttcaaatgagcagcctgaagcctgaggacacggctgtctatttctgtgcgagagatcttcgggaaatggggggacttgacttctggggccagggagccctggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaanaccctgaggtcaagttcaactggnacgtnnnggnnnnnngngcataa

SEQ ID NO: 2 - DNA, 702 bp, synthetic construct
  source 1..702: mol_type = other DNA
atgggatggtcatgtatcatcctttttctagtagcaactgcaaccggttccagatgcgacatccagatgacccagtctccatcttccgtgtctgcatctattggagacacagtcactatcacttgtcgggcgagtcagggtattcccagctgggtagcctggtatcagcagaaacctggtaaagcccctaagctcctcatttatggtgcatccaatttgcagcgtggggtcccatcaaggttcagcggcagtggatctgggacagatttcactctcaccatcagcagcctgcagcctgaagatcttgcagtttactattgtcatcagtctgacagtcttcccgggattttcggcggagggacaaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 3 - protein, 85 aa, synthetic construct
MTWVRQAPGKGLEWVSLIFAGGSTFYADSVKGRFTVSRDNSKNTLYLQMSSLKPEDTAVYFCARDLREMGGLDFWGQGALVTVSS

SEQ ID NO: 4 - protein, 104 aa, synthetic construct
MTQSPSSVSASIGDTVTITCRASQGIPSWVAWYQQKPGKAPKLLIYGASNLQRGVPSRFSGSGSGTDFTLTISSLQPEDLAVYYCHQSDSLPGIFGGGTKVEIK

SEQ ID NO: 5 - RNA, 51 nt, synthetic construct
  source 1..51: mol_type = other RNA
ggctccgggctgaacgacatcttcgaagcccagaagattgagtggcatgag

SEQ ID NO: 6 - protein, 17 aa, synthetic construct
GSGLNDIFEAQKIEWHE

SEQ ID NO: 7 - RNA, 20 nt, synthetic construct
  source 1..20: mol_type = other RNA
atgctgcaatcgtgctacaa

SEQ ID NO: 8 - RNA, 17 nt, synthetic construct
  source 1..17: mol_type = other RNA
gactgccgcctctgctc

SEQ ID NO: 9 - RNA, 11 nt, synthetic construct
  source 1..11: mol_type = other RNA
aacattgccaa

SEQ ID NO: 10 - protein, 214 aa, synthetic construct
RVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRFLRKSKLKPFERDISTEIYQVGNKPCNGVAGPNCYSPLQSYGFRPTYGVGHQPYRVVVLSFELLHSTNLVKNKCVNFN

SEQ ID NO: 11 - protein, 214 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHSTNLVKNKCVNFN

SEQ ID NO: 12 - protein, 214 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYGFRPTYGVGHQPYRVVVLSFELLHSTNLVKNKCVNFN

SEQ ID NO: 13 - protein, 214 aa, synthetic construct
RVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLLRKSKLKPFERDISTEIYQAGNKPCNGVAGPNCYSPLQSYGFRPTYGVGHQPYRVVVLSFELLHSTNLVKNKCVNFN

SEQ ID NO: 14 - protein, 213 aa, synthetic construct
RVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRLFRKSKLKPFERDISTEIYQAGNRPCKGKGPNCYFPLQSYGFRPTYGVGHQPYRVVVLSFELLHSTNLVKNKCVNFN

SEQ ID NO: 15 - protein, 214 aa, synthetic construct
RVQPTESIVRFPNITNLCPFHEVFNATTFASVYAWNRKKISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYRYRLLRKSKLKPFERDISTEIYQAGNKPCNGVAGPNCYSPLQSYGFRPTYGVGHQPYRVVVLSFELLHSTNLVKNKCVNFN

SEQ ID NO: 16 - protein, 213 aa, synthetic construct
RVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHSTNLVKNKCVNFN

SEQ ID NO: 17 - protein, 213 aa, synthetic construct
RVQPTESIVRFPNVTNLCPFHEVFNATTFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSLRKSKLKPFERDISTEIYQAGNKPCKGKGPNCYFPLQSYGFRPTYGVGHQPYRVVVLSFELLHSTNLVKNKCVNFN